(19) United States
(12) Patent Application Publication
(10) Pub. No.: US 2021/0128609 A1
(43) Pub. Date: May 6, 2021

(54) ONCOLOGY TREATMENTS USING ZINC AGENTS

(71) Applicant: XYLONIX IP HOLDINGS PTE. LTD., Singapore (SG)

(72) Inventor: Jinhyuk Fred Chung, Singapore (SG)

(21) Appl. No.: 17/252,834

(22) PCT Filed: Jun. 21, 2019
(86) PCT No.: PCT/SG2019/050308

§ 371 Date: Dec. 16, 2020

(30) Foreign Application Priority Data
Jun. 22, 2018 (SG) 10201805412T
Dec. 24, 2018 (SG) 10201811577T

(51) Int. Cl. A61K 33/30; A61K 39/395; A61K 31/315; A61K 47/64; A61K 47/55; A61P 35/00

(52) U.S. Cl. CPC A61K 33/30; A61K 39/3955; A61K 31/315; A61K 2039/505; A61K 47/551; A61P 35/00; A61K 47/645

(57) ABSTRACT

The invention relates to methods for treating a cancer patient comprising administering a Zn(II) agent or a Zn(II) agent/immune-oncology agent combination to provide a therapeutic benefit to the cancer patient. The methods are useful in treating a broad spectrum of human cancers, including solid tumors and blood-based cancerous cells. In particular embodiments, the treatment methods are directed to cancer types characterized by genetic instability mutations.

### RELATED APPLICATIONS

This application claims the benefit of and priority to Singapore Patent Application No. 10201805412T, filed Jun. 22, 2018, and Singapore Patent Application No. 10201811577T, filed Dec. 24, 2018, which applications are hereby incorporated by reference in their entirety.

### FIELD OF THE INVENTION

The invention relates to methods for treating cancer in a subject comprising administering to the subject a pharmaceutical composition comprising an effective amount of a Zn(II) agent, or, comprising effective amounts of a Zn(II) agent and an immune-oncology agent.

### BACKGROUND OF THE INVENTION

Cancer is a complex disease, wherein its clinical manifestations, symptoms, and its epidemiology widely vary in each patient according genetic factors such as gender, race, ethnicity, age group, as well as by environmental factors such as income level, education level, lifestyle, diet, etc. Although the complexity and heterogeneity of cancer has long been recognized, definitions of the disease reflect the historical development of the medical community's understanding, wherein the disease has been classified according to its physiological locations, histological appearance, and lineage.

Since the introduction of many high-throughput DNA/RNA/proteomic characterization techniques, bioinformatics, and the emergence of immune-oncology (I/O) drugs, the pharmaceutical definition of cancer may be better defined by genetic and/or epigenetic profiles. The clinical success of the precision cancer immunotherapy drugs such as imatinib, sunitinib, and pembrolizumab that target specific signaling or biochemical pathways of cancer cells, and, moreover act in a site-independent manner, are exemplary advancements that reflect this shift in the pathological definition of cancer. In one such example, the United States Food & Drug Administration approved indications of pembrolizumab and nivolumab against cancer types characterized by genetic defects known as microsatellite instability high (MSI-H) or mismatch repair deficiencies (dMMR), independent of the site of pathology.

This signals a new era of clinical oncology whereby defining cancer by its molecular biology rather than by its physio-histological features aligns the disease with the therapeutic agent's ability to deliver a therapeutic benefit. The approved I/O drugs are, however, not proving to be successful against all cancer types. For example, pembrolizumab monotherapy trial (KEYNOTE-040) for head and neck squamous cell carcinoma did not significantly improve the overall survival compared to the standard care in study of 500 patients who had not responded to one or more platinum-containing systemic treatments. Nivolumab failed as a first-line treatment for patients with non-small cell lung cancer. Atezolizumab failed in a Phase 3 trial for urothelial carcinoma despite having received accelerated approval. Also, numerous trials for combination therapies of I/O drugs with other immune-active agents have failed.

Whereas the first approved I/O drug treatments have yielded durable responses in patients, presumably by activating the adaptive immune system, and result in long-term survival, the majority of patients demonstrate resistance, while some patients relapse after initially responding. The basis for this response profile is not understood, as a full understanding of the mechanisms involved in I/O treatments is still limited. It is nonetheless recognized that there are numerous receptors and ligands involved in the immunological interactions and that the tumor microenvironment is both dynamic and evolving. One outcome of the dynamic and evolving environment is that the patient's immune system status may change over time to develop resistance to an I/O drug.

Aside from the approved I/O drugs referred to above, markers found in T lymphocytes, macrophages, and natural killer cells have been identified as targets for the development of new immunotherapy agents. A goal is to find new agents that will activate the patient's immune response against solid and hematopoietic cancers and avoid or overcome resistance to immunotherapies.

Thus, there remains an unmet need to find a composition and/or treatment method that is complementary to and/or further improves the outcome of treatments of cancers over the known I/O drug treatment methods.

Earlier applications disclosed the anticancer efficacy of digestible polymer-zinc chelate complexes (zinc γ-polyglutamate [Zn-γPGA] and zinc α-polyglutamate [Zn-αPGA], respectively) in Singapore patent application number 10201609131Y, filed 1 Nov. 2016, and Singapore patent application number 10201708886R, filed Oct. 30, 2017. Both applications are hereby incorporated by reference in their entirety.

Recognizing that these zinc(II) agents may be active themselves as tumoricidal agents and also complementary to the activity of I/O agents against cancer cells, we performed systematic research in the field and tested formulations of zinc(II) complexes as a monotherapy and in combination with a cancer immunotherapeutic agent (immune-oncology agent, or, I/O agent) against a broad spectrum of cancer types, including solid tumor cancers and blood cancers, found that such formulations have a potent tumoricidal effect and an immunotherapeutic effect, and accordingly completed our invention as described herein.

### SUMMARY OF THE INVENTION

The inventions disclosed herein are based on the surprising observation that complexes of zinc and α-polyglutamic acid (α-PGA) can induce a necrotic-like cell death in various human and mouse cancer cell lines. Without being bound by theory, detailed investigation suggests that the cell deaths have the characteristics of a necroptotic mechanism and appear to result from triggering a zinc(II)-specific PARP-1 overactivation.

In one aspect, the invention provides complexes of zinc(II) with polyglutamic acid (“Zn(II) agents,” as used generally herein) that provide a therapeutic benefit against solid or hematopoietic cancer cells, including, for example, solid tumors in human patients. The Zn(II) agents demonstrate a more consistent and broader cytotoxicity than cisplatin while also demonstrating less sensitivity to conventional drug resistance mutations. The Zn(II) agents also elicit a pan-immunity stimulatory effect.

In another aspect, the polyglutamic acid used in the Zn(II) agent is conjugated with tumor-targeting ligands to further enhance therapeutic efficacy. Whether conjugated or not, in some embodiments the polyglutamic acid is the gamma (γ) form of the polymer, while in other embodiments it is the alpha (α) form.

In another aspect, the invention provides methods of treating a patient with a solid or hematopoietic cancer. In another aspect, the invention provides methods of enhancing immune-oncology treatments of patients with cancer by treating such patients with Zn(II) agents in combination with the immune-oncology treatment.

In another aspect, the invention provides methods for treating a patient with a tumor that includes tumor cells that have genetic instability mutations and/or genetic instability due to gene overexpression. In some embodiments, the genetic instability mutations of the tumor cells described herein are dysfunctional mutations in one or more genes selected from ATM; ATR; PAXIP1; BRCA1; BRCA2; WRN; RFC1; RPA1; ERCC1; ERCC4; ERCC6; MGMT; PARP1; PARP2; NEIL3; XRCC1; MLH1; PMS2; TP53; CREBBP; JAK1; NFKB1; MSH2; MSH3; MSH6; and MLH3. In some embodiments, the dysfunctional mutation is in the ATM gene. In some embodiments, the dysfunctional mutation is in the ATR gene. In some embodiments, the dysfunctional mutation is in the PAXIP1 gene. In some embodiments, the dysfunctional mutation is in the BRCA1 gene. In some embodiments, the dysfunctional mutation is in the BRCA2 gene. In some embodiments, the dysfunctional mutation is in the WRN gene. In some embodiments, the dysfunctional mutation is in the RFC1 gene. In some embodiments, the dysfunctional mutation is in the RPA1 gene. In some embodiments, the dysfunctional mutation is in the ERCC1 gene. In some embodiments, the dysfunctional mutation is in the ERCC4 gene. In some embodiments, the dysfunctional mutation is in the ERCC6 gene. In some embodiments, the dysfunctional mutation is in the MGMT gene. In some embodiments, the dysfunctional mutation is in the PARP1 gene. In some embodiments, the dysfunctional mutation is in the PARP2 gene. In some embodiments, the dysfunctional mutation is in the NEIL3 gene. In some embodiments, the dysfunctional mutation is in the XRCC1 gene. In some embodiments, the dysfunctional mutation is in the MLH1 gene. In some embodiments, the dysfunctional mutation is in the PMS2 gene. In some embodiments, the dysfunctional mutation is in the TP53 gene. In some embodiments, the dysfunctional mutation is in the CREBBP gene. In some embodiments, the dysfunctional mutation is in the JAK1 gene. In some embodiments, the dysfunctional mutation is in the NFKB1 gene. In some embodiments, the dysfunctional mutation is in the MSH2 gene. In some embodiments, the dysfunctional mutation is in the MSH3 gene. In some embodiments, the dysfunctional mutation is in the MSH6 gene. In some embodiments, the dysfunctional mutation is in the MLH3 gene.

Accordingly, in some embodiments, therapeutically effective amounts of Zn(II) agents are administered in monotherapy treatment methods.

In some embodiments, therapeutically effective amounts of any of the Zn(II) agents described herein are administered in combination with therapeutically effective amounts of immune-oncology agents in combination treatment methods. One embodiment of a method for treating a tumor in a patient comprises administering therapeutically effective amounts of (i) a Zn(II)/γ-polyglutamic acid composition and/or a Zn(II)/α-polyglutamic acid composition, in combination with (ii) an immune-oncology agent that targets a T-lymphocyte marker, a macrophage marker, or a natural killer cell marker.

In various embodiments, the immune-oncology agent is an immune checkpoint inhibitor. Non-limiting examples of immune checkpoint inhibitors include PD-1 inhibitors, PD-L1 inhibitors, CTLA-4 inhibitors. Additional non-limiting examples of immunomodulatory inhibitors include LAG-3 inhibitors, TIM-3 inhibitors, TIGIT inhibitors, B7-H3 inhibitors, VISTA inhibitors, ICOS inhibitors, CD27 inhibitors, GITR inhibitors, CD47 inhibitors, IDO inhibitors, KIR inhibitors, and CD94/NKG2A inhibitors.

Further embodiments of the invention provide for combination therapies that comprise any of the Zn(II) agents disclosed herein with two immunotherapy agents. In some embodiments, the first and second immunotherapy agents are each independently selected from PD-1 inhibitors, PD-L1 inhibitors, CTLA-4 inhibitors, LAG-3 inhibitors, TIM-3 inhibitors, TIGIT inhibitors, B7-H3 inhibitors, VISTA inhibitors, ICOS inhibitors, CD27 inhibitors, and GITR inhibitors. In many embodiments, the above first and second immunotherapy agents are from different classes of inhibitors, that is, they target different markers.

In other embodiments using two immunotherapy agents, a first agent is selected from PD-1 inhibitors, PD-L1 inhibitors, CTLA-4 inhibitors, LAG-3 inhibitors, TIM-3 inhibitors, TIGIT inhibitors, B7-H3 inhibitors, VISTA inhibitors, ICOS inhibitors, CD27 inhibitors, and GITR inhibitors, and a second agent is selected from CD47 inhibitors and IDO inhibitors. In other embodiments using two immunotherapy agents, a first agent is selected from PD-1 inhibitors, PD-L1 inhibitors, CTLA-4 inhibitors, LAG-3 inhibitors, TIM-3 inhibitors, TIGIT inhibitors, B7-H3 inhibitors, VISTA inhibitors, ICOS inhibitors, CD27 inhibitors, and GITR inhibitors, and a second agent is selected from KIR inhibitors and CD94/NKG2A inhibitors.

### BRIEF DESCRIPTION OF THE FIGURES

FIG. 1 illustrates certain embodiments of Zn(II) agents according to the invention.

FIGS. 2A-2B show the results of cell viability assays upon treating HeLa cells with certain embodiments of Zn(II) agents.

FIGS. 3A-3B show the results of assays to determine IC50 values for certain embodiments of Zn(II) agents.

FIGS. 4A-4E show the results of experiments indicative of the cell death mechanism observed when treating cells with an embodiment of a Zn(II) agent.

FIGS. 5A-5I show the dose-response curves for the 50 cell line study described in Example 6.

FIG. 6 shows a table with the IC50 screening data and the mutated genes identified for each cell type for the 50 cell line study described in Example 6.

FIGS. 7A-7B show an analysis of the response to treatment in the 50 cell line study described in Example 6.

FIG. 8 shows single gene mutation effects on drug sensitivity in the 50 cell line study described in Example 6.

FIGS. 9A-9F show an analysis of the response to treatment in the 50 cell line study described in Example 6.

FIGS. 10A-10P show multiple gene mutation effects on drug sensitivity in the 50 cell line study described in Example 6.

FIGS. 11A-11D show an analysis of APOBEC3B gene mutation and effect of its overexpression on IC50 value distribution for C004 and cisplatin.

FIGS. 12A-12C show candidate biomarkers.

FIG. 13 shows results from a repeat toxicity test of C004.

FIGS. 14A-14B show results from administration of C005D.

FIG. 15 shows results from C005D monotherapy and C005D/anti-PD-1 mAb combination therapy treatments.

FIG. 16 shows the gating strategy for in vivo immunity characterization for the treatment study described in Example 10.

FIGS. 17A-17B show an analysis of the immunological effects of the treatments described in Example 10.

FIGS. 17C-17G show the results of the HCC PDX-HuMice model test and TIL analyses described in Example 11.

FIG. 18 shows a mechanism of the PARP-1 overdrive mediated necrotic cytotoxicity of Zn(II) agents according to embodiments of the invention.

FIG. 19 shows a schematic representation of the inferred tumorical mechanism of the Zn(II) agents according to embodiments of the invention.

### EXEMPLARY EMBODIMENTS

Embodiments of the present invention includes, but are not limited to, the following:

1. A method for treating a patient with a tumor comprising administering to said patient a therapeutically effective amount of a Zn(II) agent.
2. A method for treating a patient with a tumor comprising administering to said patient a therapeutically effective amount of a Zn(II) agent in combination with an immune-oncology agent.
3. The method according to embodiment 2, wherein said immune-oncology agent is an immune checkpoint inhibitor.
4. The method according to embodiment 3, wherein said immune checkpoint inhibitor is an anti-cytotoxic T-lymphocyte antigen-4 (CTLA-4) antibody or an antigen-binding portion thereof that binds specifically to CTLA-4 and inhibits CTLA-4 activity; or a programmed cell death-1 (PD-1) antibody or an antigen-binding portion thereof that binds specifically to a PD-1 receptor and inhibits PD-1 activity.
5. The method according to any one of embodiments 1 to 4, wherein said tumor includes tumor cells that have genetic instability mutations and/or genetic instability due to gene overexpression.
6. The method according to embodiment 5, wherein said genetic instability mutations are dysfunctional mutations in one or more genes selected from ATM; ATR; PAXIP1; BRCA1; BRCA2; WRN; RFC1; RPA1; ERCC1; ERCC4; ERCC6; MGMT; PARP1; PARP2; NEIL3; XRCC1; MLH1; PMS2; TP53; CREBBP; JAK1; NFKB1; MSH2; MSH3; MSH6; and MLH3.
7. A method for increasing the tumor infiltrating leukocyte population of CD4+ T cells and CD8+ T cells in a tumor in a patient comprising administering to said patient having said tumor a therapeutically effective amount of a Zn(II) agent.
8. A method for increasing the tumor infiltrating leukocyte population of CD4+ T cells and CD8+ T cells in a tumor in a patient comprising administering to said patient having said tumor a therapeutically effective amount of a Zn(II) agent in combination with an immune-oncology agent.
9. The method according to embodiment 8, wherein said immune-oncology agent is an immune checkpoint inhibitor.
10. The method according to embodiment 8, wherein said immune checkpoint inhibitor is an anti-cytotoxic T-lymphocyte antigen-4 (CTLA-4) antibody or an antigen-binding portion thereof that binds specifically to CTLA-4 and inhibits CTLA-4 activity; or a programmed cell death-1 (PD-1) antibody or an antigen-binding portion thereof that binds specifically to a PD-1 receptor and inhibits PD-1 activity.
11. The method according to any one of embodiments 1-10, wherein said Zn(II) agent comprises Zn(II)/γ-polyglutamic acid and/or Zn(II)/α-polyglutamic acid.
12. A method for treating a tumor in a patient, comprising administering a therapeutically effective amount of (i) a Zn(II)/polyglutamic acid agent in combination with (ii) an immune-oncology agent that targets a T-lymphocyte marker, a macrophage marker, or a natural killer cell marker.
13. The method of embodiment 12, wherein the T-lymphocyte marker is lymphocyte activation gene 3 (LAG-3).
14. The method of embodiment 12, wherein the T-lymphocyte marker is T-cell immunoglobulin- and mucin-domain-containing molecule 3 (TIM-3).
15. The method of embodiment 12, wherein the T-lymphocyte marker is T-cell immunoglobulin and ITIM domain (TIGIT).
16. The method of embodiment 12, wherein the T-lymphocyte marker is B7-H3 (CD276).
17. The method of embodiment 12, wherein the T-lymphocyte marker is V-domain containing Ig suppressor of T-cell activation (VISTA).
18. The method of embodiment 12, wherein the T-lymphocyte marker is inducible T-cell costimulator (ICOS).
19. The method of embodiment 12, wherein the T-lymphocyte marker is CD27.
20. The method of embodiment 12, wherein the T-lymphocyte marker is glucocorticoid-induced TNF receptor (GITR).
21. The method of embodiment 12, wherein the macrophage marker is CD47.
22. The method of embodiment 12, wherein the macrophage marker is indoleamine-2,3-dioxygenase (IDO).
23. The method of embodiment 12, wherein the natural killer cell marker is killer immunoglobulin-like receptor (KIR).
24. The method of embodiment 12, wherein the natural killer cell marker is CD94/NKG2A.
25. The method of any one of embodiments 12 to 24, wherein said Zn(II)/polyglutamic acid agent comprises polyglutamic acid conjugated to a tumor-targeting moiety and/or a charge-carrying moiety.
26. The method of embodiment 25, wherein said polyglutamic acid conjugated to a tumor-targeting moiety and/or a charge-carrying moiety is γ-polyglutamic acid.
27. The method of embodiment 25, wherein the molecular weight of said polyglutamic acid is in the range of about 2.5 kDa to about 60 kDa.
28. The method according to embodiment 5, wherein said genetic instability due to overexpression is caused by overexpression of APOBEC3B.
29. The method according to embodiment 6, wherein said gene is ATM.
30. The method according to embodiment 6, wherein said gene is ATR.
31. The method according to embodiment 6, wherein said gene is PAXIP1.
32. The method according to embodiment 6, wherein said gene is BRCA1.
33. The method according to embodiment 6, wherein said gene is BRCA2.
34. The method according to embodiment 6, wherein said gene is WRN.
35. The method according to embodiment 6, wherein said gene is RFC1.
36. The method according to embodiment 6, wherein said gene is RPA1.
37. The method according to embodiment 6, wherein said gene is ERCC1.
38. The method according to embodiment 6, wherein said gene is ERCC4.
39. The method according to embodiment 6, wherein said gene is ERCC6.
40. The method according to embodiment 6, wherein said gene is MGMT.
41. The method according to embodiment 6, wherein said gene is PARP1.
42. The method according to embodiment 6, wherein said gene is PARP2.
43. The method according to embodiment 6, wherein said gene is NEIL3.
44. The method according to embodiment 6, wherein said gene is XRCC1.
45. The method according to embodiment 6, wherein said gene is MLH1.
46. The method according to embodiment 6, wherein said gene is PMS2.
47. The method according to embodiment 6, wherein said gene is TP53.
48. The method according to embodiment 6, wherein said gene is CREBBP.
49. The method according to embodiment 6, wherein said gene is JAK1.
50. The method according to embodiment 6, wherein said gene is NFKB1.
51. The method according to embodiment 6, wherein said gene is MSH2.
52. The method according to embodiment 6, wherein said gene is MSH3.
53. The method according to embodiment 6, wherein said gene is MSH6.
54. The method according to embodiment 6, wherein said gene is MLH3.

### DETAILED DESCRIPTION OF THE INVENTION

The meaning of abbreviations used herein is as follows: “kDa” means kiloDalton; “wt %” means percent by weight.

### Zn(II) Agents

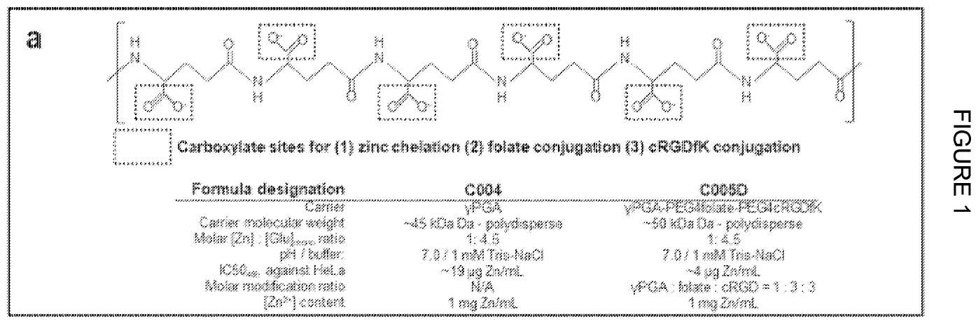

Zn(II) agents are comprised of zinc(II) (equivalently, Zn²⁺) complexed to polyglutamic acid. A generalized structure of an embodiment of a Zn(II) agent in shown in FIG. 1. Polyglutamic acid (“PGA”) is a condensed polymer of glutamic acid, which, because it contains two carboxylic acids may form in two configurations. Condensation via the α-carboxylate moiety yields α-polyglutamic acid, and via the γ-carboxylate moiety yields γ-polyglutamic acid. Zn(II) agents may be prepared with α-PGA, γ-PGA, or both α-PGA and γ-PGA, and any such composition may also be referred to as “ZnPGA.” It should be understood that if the form (α- or γ-) is not specified then either form, separately, or both forms, as a blend in any ratio, may be inferred, unless otherwise specified. ZnPGA compositions are generally purified such that free Zn(II) ions as well as the original counterions to the Zn cation are removed in the process.

Zinc salts used to prepare Zn(II) agents are zinc(II) salts (equivalently, Zn²⁺ salts), wherein the counterion (anion) may be any inorganic or organic anion suitable for use in the manufacture of a pharmaceutical product. Suitable anions are those that are tolerated by the human body, including those that are not toxic. Generally, the zinc salt can be represented by the formulas Zn²⁺X²⁻ or Zn²⁺(X⁻)₂ or even Zn²⁺(X⁻)(Y⁻), where X and Y are suitable anions. The anion may be selected from the group of anions that are a component of an FDA-approved pharmaceutical product. In some embodiments, the zinc(II) salt is a pharmaceutically acceptable zinc salt. Examples of zinc salts include zinc chloride, zinc sulfate, zinc citrate, zinc acetate, zinc picolinate, zinc gluconate, amino acid-zinc chelates, such as zinc glycinate, or other amino acids known and used in the art.

Alpha-polyglutamic acid (alternatively α-polyglutamic acid or α-PGA) is a polymer of glutamic acid, an amino acid, where the polymer backbone is formed by a peptide bond joining the amino group and carboxyl group at the α-carbon (the typical peptide bond formed in proteins), not the carboxyl group in the amino acid side chain. α-PGA can be formed from the L isomer, the D isomer, or the DL racemate of glutamic acid. Any of these forms may be used, and two or more different forms may be used together in any proportion. The various isomeric forms of α-PGA may be synthetic or derived from natural sources. Whereas organisms usually only produce poly(amino acids) from the L isomer, certain bacterial enzymes that produce α-PGA can produce polymers from either isomer or both isomers.

Gamma-polyglutamic acid (alternatively γ-polyglutamic acid or γ-PGA) is a polymer of glutamic acid, an amino acid, where the polymer backbone is formed by a peptide bond joining the amino group and carboxyl group in the amino acid side chain (at the γ-carbon). γ-PGA can be formed from the L isomer, the D isomer, or the DL racemate of glutamic acid. Any of these forms may be used, and two or more different forms may be used together in any proportion. The various isomeric forms of γ-PGA may be synthetic or derived from natural sources. γ-PGA is found, for example, in Japanese natto and in sea kelp. Whereas organisms usually only produce poly(amino acids) from the L isomer, certain bacterial enzymes that produce γ-PGA can produce polymers from either isomer or both isomers.

α-PGA and γ-PGA of various sizes and various polymer dispersities may be used, and the same considerations apply to each. The polymer molecular weight of PGA is generally at least about 1 kDa and at most about 100 kDa. In some embodiments, the polymer molecular weight of PGA is at least about 1 kDa, or at least about 2.5 kDa, or at least about 5 kDa, or least about 10 kDa, or at least about 20 kDa, or least about 30 kDa, or at least about 35 kDa, or at least about 40 kDa, or at least about 50 kDa. In some embodiments, the polymer molecular weight of PGA is at most about 100 kDa, or at most about 90 kDa, or at most about 80 kDa, or at most about 70 kDa, or at most about 60 kDa. An acceptable polymer molecular weight range may be selected from any of the above indicated polymer molecular weight values. In an embodiment, the polymer molecule weight is in the range of about 2.5 kDa to about 50 kDa. In an embodiment, the polymer molecule weight is in the range of about 50 kDa to about 100 kDa. In one embodiment, the polymer molecular weight is about 50 kDA. Polymer molecular weights are typically given as a number average molecular weight (Mn) based on a measurement by gel permeation chromatography (GPC). The above polymer masses are cited as Mn; other measurement techniques can be used to determine, e.g., a mass (weight) average molecular weight (Mw), and the specification for any given polymer can be converted among the various polymer mass representations.

PGA may comprise tumor-targeting moieties. Such moieties may be selected from folic acid, N⁵,N¹⁰-dimethyl tetrahydrofolate (DMTHF), and RGD peptide. Each of said moieties may be covalently joined to polyglutamic acid in any combination and ratio to form, e.g., a folate conjugate and/or a DMTHF conjugate and/or an RGD peptide conjugate of PGA.

Folate receptor protein is often expressed in many human tumors. Folates naturally have a high affinity for the folate receptors, and further, upon binding, the folate and the attached conjugate may be transported into the cell by endocytosis. In this way, a ZnPGA modified with folic acid can target and accumulate at tumor cells and deliver zinc(II) to the vicinity of and/or inside the tumor cells.

DMTHF is also known to have a high affinity for folate receptors. The preparation of DMTHF is described in Leamon, C. P. et al., Bioconjugate Chemistry 13, 1200-1210. Furthermore, there are two major isoforms of the folate receptor (FR), FR-α and FR-β and DMTHF has been shown to have a higher affinity for FR-α over FR-β (Vaitilingam, B., et al., The Journal of Nuclear Medicine 53, 1127-1134.). This is beneficial for targeting tumor cells because FR-α is overexpressed in many malignant cell types, whereas FR-β is overexpressed on macrophages associated with inflammatory disease, Thus, conjugating DMTHF to PGA provides a conjugate that may selectively bind to folate receptors expressed by tumor cells.

Similarly, RGD peptides are known to bind strongly to α(V)β(3) integrins, which are expressed on tumoral endothelial cells as well as on some tumor cells. Thus, RGD conjugates may be used for targeting and delivering antitumor agents to the tumor site.

As contemplated in this invention, PGA may be conjugated (i.e., covalently bound) with any one or two, or all of these tumor targeting agents, and when two or more are present, the relative ratio of these agents is not particularly limited. For example, a PGA carrier may comprise a conjugate of PGA with (a) folic acid, (b) DMTHF, (c) RGD, (d) folic acid and DMTHF, (e) folic acid and RGD, (f) DMTHF and RGD, or (g) folic acid, DMTHF, and RGD. Other similar tumor-targeting moieties known to those of skill in the art are also within the scope of the invention.

α-PGA has a free carboxylic acid group at the γ-carbon of each glutamic acid unit and γ-PGA has a free carboxylic acid group at the α-carbon of each glutamic acid unit that can be used to form conjugates with folic acid and with RGD peptide. Folic acid has an exocyclic amine group that may be coupled with the free carboxylic acid group of glutamic acid to form an amide bond joining the two. The same exocyclic amine group as in folic acid is available in DMTHF for amide bond formation. RGD conjugates are also well-known in the art, and can also be similarly covalently joined to the free carboxylic acid group via, for example, the free α-amino group in RGD. Alternatively, either moiety may be conjugated to PGA via a spacer group, such as, for example, polyethylene glycol amine. Examples of conjugation reactions between the γ-carbon carboxylate group of α-PGA and an amino group can be found in U.S. Pat. No. 9,636,411 to Bai et al. and with an amino and hydroxyl group can be found in U.S. Pre-Grant Publication No. 2008/0279778 by Van et al. Examples of conjugation reactions to γ-PGA, including that of folic acid and citric acid, can be found in WO 2014/155142 (published Oct. 2, 2014).

PGA may comprise charge-modifying moieties. Such moieties may be selected from citric acid, ethylenediamine tetraacetic acid (EDTA), 1,4,7,10-tetracyclododecane-N, N′,N″, N′″-tetraacetic acid (DOTA), and diethylenetriamine pentaacetic acid (DTPA). Any combination of said moieties may be covalently joined to polyglutamic acid, again, at the free carboxylic acid, as discussed above. Citric acid may be conjugated to the free carboxylic acid group of PGA by forming an ester linkage. (See, e.g., WO 2014/155142 for a reaction involving the α-carbon of γ-PGA.) EDTA, DOTA, and DTPA may be joined to PGA using, for example, spacer groups to join the amines of these moieties to the free carboxylic acid group of PGA. Numerous options are available to one of skill in the art. The charge-modifying moieties can be used as sites for chelating Zn(II) ions, and the charge-modification will also affect transport and solubility of the ZnPGA complexes and as such can be used to tune the pharmaceutical effects of the carrier and the ZnPGA complexes.

PGA may comprise both tumor-targeting and charge-modifying moieties so that the benefits and functionality of both types of moieties may be imparted to the PGA carrier, and to the Zn(II) agent. Any combination of the tumor-targeting and charge-modifying moieties may be conjugated to PGA, and the relative ratio of the moieties is not particularly limited.

The amount of zinc ion in the Zn(II) agents according to the invention may be expressed as a ratio of zinc to glutamic acid units (“GAU”). The same concept can be used when chelating groups are conjugated to PGA, through the relationship of the average number of chelating sites provided per glutamic acid unit. The ratio may be as high as 1:1 Zn:GAU, though this nominally precludes conjugated tumor-targeting or charge-modifying moieties. Lower ratios of 1:2, 1:5, 1:10, 1:20, are contemplated, and even lower ratios are possible, but then the amount of PGA included in a dosage amount needed to deliver a suitable dose of zinc(II) increases. An appropriate balance between the dosage amount and amount of non-zinc component can be determined by one of ordinary skill in the art. In one embodiment, the ratio is any value between about 1:2 and about 1:10 Zn:GAU. In another embodiment, the ratio is any value between about 1:3 and about 1:6. In another embodiment, the ratio is about 1:4.5.

The number of tumor-targeting and charge-modifying moieties in the Zn(II) agents is usually expressed as the average number of conjugated moieties per PGA polymer. Analytical techniques for determining the average number of moieties bound per polymer strand are known to those of ordinary skill in the art. The desired number of moieties per polymer strand reflects a balance between the average polymer size and thus the number of monomeric units available, the desired ratio of Zn:GAU, the desired number of different types of moieties, and the like. Ratios of about 1:1, about 2:1, about 3:1, about 4:1, about 5:1, about 6:1, or about 8:1 of any single moiety per polymer strand are contemplated. Higher ratios are also contemplated, however, one of ordinary skill in the art recognizes that there is often less benefit gained as more of a moiety is added.

ZnPGA compositions are generally prepared by first obtaining or preparing a PGA polymer having the desired average molecular weight, polydispersity, conjugated moieties, and the like. One may also use a mixture of PGA polymers wherein the characteristics of each differ, in order to, for example, provide a broader range of polymer sizes, provide different targeting capabilities, and the like. Then, the PGA polymer is combined with a zinc salt in buffered aqueous solution and suitably processed for preparing a pharmaceutically acceptable formulated composition, a Zn(II) agent, that may be used in the treatment methods disclosed herein. Exemplary methods for preparing ZnPGA compositions and Zn(II) agents (formulations) are provided in the examples herein.

The concentration of zinc provided in a composition or formulation in a liquid dosage form is generally in the range of about 1 μg/mL to about 100 mg/mL of zinc (zinc(II) ion). This corresponds to a range of about 0.0001 wt % to about 10 wt % of zinc. The concentration of Zn(II) may be at least about 10 μg/mL, or at least about 0.1 mg/mL, or at least about 1 mg/mL, or at least about 10 mg/mL, or at least about 50 mg/mL, or the range for the concentration of Zn(II) may fall within any two of these exemplary concentrations. In one embodiment, the concentration may be in the range of about 100 μg/mL to about 5 mg/mL. In another embodiment, the concentration may be in the range of about 200 μg/mL about 2 mg/m L.

The amount of the liquid provided in the dosage form will determine the total dosage amount. For example, 100 mL amount of liquid would provide about 10 mg to about 500 mg of Zn(II) for the first exemplary range above and about 20 mg to about 200 mg of Zn(II) for the second range. The appropriate concentration and amount of the liquid formulation to use will generally depend on the weight of the patient. The appropriate administration regimen is generally provided as an amount of zinc per patient body weight (e.g., per kg) per day, and thus expressed as the number of mg Zn/kg/day.

Suitable liquid formulations include a liquid solution, a liquid suspension, a syrup, and an oral spray. The liquid solutions can be administered orally or administered by injection, such intravenously, intradermally, intramuscularly, intrathecally, or subcutaneously, or directly into or in the vicinity of a tumor, whereas liquid suspensions, syrups and sprays are generally appropriate for oral administration.

Methods for preparing liquid dosage forms comprises mixing together the desired amounts of (i) zinc salt(s) and PGA carrier and/or (ii) a ZnPGA complex, along with suitable excipients. Some embodiments further comprise a gastro-resistant binder and/or coating in the formulation.

A liquid solution formulation may be prepared with suitable carriers, diluents, buffers, preservatives, or other excipients suitably selected with regard to the form of administration. For example, intravenous formulations may be prepared buffered at a suitable pH and with isotonicity agents.

An embodiment of a liquid formulation suitable for injection or oral delivery comprises a zinc(II) salt, PGA carrier (unmodified PGA and/or any forms of modified PGA, as described above), and water. In further embodiments, the liquid formulation may further comprise a buffer and/or a salt, such as sodium chloride. When a buffering agent is included, a preferred buffering pH is in the range of about pH 4 to about pH 9. When injected, preferably the solution is isotonic with the solution into which it is to be injected and of suitable pH. In one embodiment, zinc sulfate heptahydrate, α-PGA, and sodium chloride are combined in water, wherein the concentration of zinc(II) is 1 mg/mL and γ-PGA is 10 mg/mL. The polymer molecular weight of γ-PGA may be selected from any of the ranges described above. In one embodiment, it is in the range of about 1 kDa to about 100 kDa, and in other embodiments it is in the range of about 2.5 kDa to about 50 kDa. In any embodiment, one or more polymer molecular weight forms of γ-PGA may be included.

In some embodiments, a zinc salt and a PGA carrier may be prepared as a ZnPGA complex. Generally, to form a ZnPGA complex the zinc salt(s) and PGA carrier are combined and purified as described, for example, in Examples 1, 2, and 12-23. The solution of the obtained ZnPGA complex may be diluted or substantially dried and reconstituted in more concentrated form for use in the procedure for preparing a liquid dosage form. ZnPGA complexes may be formulated as injectable solutions, or as a liquid suspension, syrup, or spray.

As disclosed in Example 9, mice were treated by injection with solutions of C004 Zn(II) agent and received a physiologically relevant dose of 0.5, 1.0, or 2.5 mg/kg body weight/day of Zn(II), or by injection with solution of C005D Zn(II) agent for a physiologically relevant dose of 0.5, 1.0, or 2.0 mg/kg body weight/day of Zn(II).

As disclosed in Example 10, mice were treated intravenously with 2 mg/kg body weight/day of C005D solution alone or in conjunction with anti-human PD-1 antibody in a combination therapy treatment.

In some embodiments the Zn(II) agent is formulated as a solid dosage form. The amount of zinc included in a single solid dosage form is generally in the range of about 1 to about 100 mg of zinc (zinc(II) ion). Thus, the particular amount of zinc salt(s) used in a formulated composition will be higher because amount of the salt must account for the weight of the counterion. Considering only zinc(II), the amount provided in a dosage form may be up to about 100 mg, up to about 75 mg, up to about 50 mg, up to about 25 mg, up to about 10 mg of zinc, or up to about 5 mg. The amount of zinc(II) provided in a solid dosage form is generally at least about 1 mg. By way of comparison, commonly available supplements provide, for example, 20, 25, 30, 50, 75, and even 100 mg of zinc. Any amount of zinc in this range, or even higher, is acceptable so long as the amount provided does not cause physiologically excessive levels of zinc to be absorbed. What might be considered an excessive level and the risk therefrom, however, is to be balanced against the therapeutic benefit gained by treating a tumor. Although a tolerable upper intake level of zinc in most adults is about 40 mg/day (and for children it is lower), it should be recognized that all of the zinc in the solid dosage form taken orally is unlikely to be absorbed; some of it will pass through the body without being adsorbed. Because the amount of zinc absorbed will also vary with the formulation, the upper limit for zinc content in a particular formulation can be tested by methods known in the art to ascertain the level of uptake provided by the formulation, and then in view of any therapeutic benefit in the treatment gained by administering the formulation, one may adjust the amount administered for a given dosage form or formulation accordingly.

In other embodiments, zinc(II) may also be provided from a solid suspended in liquid. The amount of zinc(II) and the volume of the suspension provided follows the guidance set out above for solid and liquid dosage forms.

The amount of PGA included in a liquid dosage form is generally in the range of about 0.01 wt % to about 10 wt %. In some embodiments the amount is about 0.1 wt % or about 1 wt %.

The amount used is generally based upon the desired molar ratio between zinc and polyglutamic acid monomer units, the nature of the carrier PGA (that is whether it is unmodified, or modified with a tumor-targeting moieties and/or a charge-modifying moieties), and the degree of formation of Zn(II) complexes with the carrier PGA. For example, as illustrated in Examples 12 and 13, ZnPGA complexes were obtained as solutions comprising approximately 1 wt % PGA with approximately 400 μg/mL of complexed zinc.

The amount of PGA included in a solid dosage form is generally in the range of about 10 wt % to about 40 wt %. In some embodiments the amount is about 20 wt % or about 30 wt %. The amount used is generally based upon the desired molar ratio between zinc and polyglutamic acid monomer units, the mass of the zinc salt (accounting for the weight of the counterion), and the amount of excipients needed to provide an acceptable formulated dosage form. For example, the greater the amount of PGA and zinc salt used, the lesser the amount of excipients that can be added for a given overall dosage form size. Those of skill in the art can readily balance the amount of active ingredients versus the amount and type of excipients needed to obtain stable dosage forms. The desired ratio between zinc and PGA can also be expressed as a ratio of milligrams of zinc to wt % of PGA per dosage form. Exemplary ratios include 5 mg:10 wt %; 5 mg: 20 wt %; 5 mg: 40 wt %; 30 mg:10 wt %; 30 mg: 20 wt %; 30 mg: 40 wt %; or even 100 mg:10 wt %; 100 mg: 20 wt %; 100 mg: 40 wt %; or any other sets of values apparent from the values cited for each ingredient.

To arrive at suitable solid or liquid compositions and pharmaceutically acceptable formulations having an effective amount of a Zn(II) salt and a polyglutamic acid carrier, the relative amounts and the respective concentrations of PGA and zinc can be adjusted readily by those of skill in the art in accordance with the disclosure.

The dosage forms described herein may be administered to provide a therapeutically effective amount of zinc(II) to achieve the desired biological response in a subject. A therapeutically effective amount means that the amount of zinc delivered to the patient in need of treatment through the combined effects of the Zn(II), the PGA, and any modifications to the PGA, the form of any ZnPGA complex, and/or the delivery efficiency of the dosage form, and the like, will achieve the desired biological response. The therapeutically effective amount may also differ in combination therapy treatment methods wherein the patient also is receiving immune-oncology agents. Where synergistic effects are obtained, a therapeutically effective amount of the zinc(II) agent may be lower than for a monotherapy treatment method.

The desired biological response include the prevention of the onset or development of a tumor or cancer, the partial or total prevention, delay, or inhibition of the progression of a tumor or cancer, or the prevention, delay, or inhibition of the recurrence of a tumor or cancer in the subject, such as a mammal, such as in a human (also may be referred to as a patient). Clinical benefits of the treatment methods can be assessed by objective response rate, tumor size, duration of response, time to treatment failure, progression free survival, and other primary and secondary endpoints assessed in clinical use.

The methods of treatments disclosed herein can be used for the treatment of a broad spectrum of human cancers, such as solid or hematopoietic cancers or tumors. For instance, the methods and treatments disclosed herein can be used in treating a patient with a tumor that includes tumor cells that have genetic instability mutations and/or genetic instability due to gene overexpression. In some embodiments, the genetic instability mutations of the tumor cells described herein are dysfunctional mutations in one or more genes selected from ATM; ATR; PAXIP1; BRCA1; BRCA2; WRN; RFC1; RPA1; ERCC1; ERCC4; ERCC6; MGMT; PARP1; PARP2; NEIL3; XRCC1; MLH1; PMS2; TP53; CREBBP; JAK1; NFKB1; MSH2; MSH3; MSH6; and MLH3. In some embodiments, the dysfunctional mutation is in the ATM gene. In some embodiments, the dysfunctional mutation is in the ATR gene. In some embodiments, the dysfunctional mutation is in the PAXIP1 gene. In some embodiments, the dysfunctional mutation is in the BRCA1 gene. In some embodiments, the dysfunctional mutation is in the BRCA2 gene. In some embodiments, the dysfunctional mutation is in the WRN gene. In some embodiments, the dysfunctional mutation is in the RFC1 gene. In some embodiments, the dysfunctional mutation is in the RPA1 gene. In some embodiments, the dysfunctional mutation is in the ERCC1 gene. In some embodiments, the dysfunctional mutation is in the ERCC4 gene. In some embodiments, the dysfunctional mutation is in the ERCC6 gene. In some embodiments, the dysfunctional mutation is in the MGMT gene. In some embodiments, the dysfunctional mutation is in the PARP1 gene. In some embodiments, the dysfunctional mutation is in the PARP2 gene. In some embodiments, the dysfunctional mutation is in the NEIL3 gene. In some embodiments, the dysfunctional mutation is in the XRCC1 gene. In some embodiments, the dysfunctional mutation is in the MLH1 gene. In some embodiments, the dysfunctional mutation is in the PMS2 gene. In some embodiments, the dysfunctional mutation is in the TP53 gene. In some embodiments, the dysfunctional mutation is in the CREBBP gene. In some embodiments, the dysfunctional mutation is in the JAK1 gene. In some embodiments, the dysfunctional mutation is in the NFKB1 gene. In some embodiments, the dysfunctional mutation is in the MSH2 gene. In some embodiments, the dysfunctional mutation is in the MSH3 gene. In some embodiments, the dysfunctional mutation is in the MSH6 gene. In some embodiments, the dysfunctional mutation is in the MLH3 gene.

Moreover, all tumor types that are susceptible to PARP1-mediated necrosis are contemplated to be indications that can be treated according to the methods of treatment disclosed herein. The various examples demonstrate the efficacy of treatments according to embodiments of the disclosed methods using embodiments of the disclosed compositions and pharmaceutical formulations. The results demonstrate effective treatments of mouse cancer cells and human cancer cells in vivo, in mice models, including in humanized immunity mice.

Achieving a therapeutically effective amount will depend on the formulation's characteristics, any will vary by gender, age, condition, and genetic makeup of each individual. Individuals with inadequate zinc due to, for example, genetic causes or other causes of malabsorption or severe dietary restriction may require a different amount for therapeutic effect compared to those with generally adequate levels of zinc.

The subject is generally administered an amount of zinc from about 0.1 mg/kg/day up to about 5 mg/kg/day. In some embodiments, the amount of zin administered is from about 1.0 mg/kg/day to about 3 mg/kg/day. Multiple dosage forms may be taken together or separately in the day. The oral dosage forms generally may be administered without regard to meal time. Treatment generally continues until the desired therapeutic effect is achieved. Low dosage levels of the compositions and formulations described herein may also be continued as a treatment according to an embodiment of the invention if a tumor regresses or is inhibiting, for the purpose of preventing, delaying, or inhibiting its recurrence, or used as a preventative treatment.

### Immune-Oncology Agents

The immune-oncology agents contemplated for use in combination therapies with the above-mentioned Zn(II) agents may be any cancer immunotherapy agent. The terms “immune-oncology agent,” “cancer immunotherapeutic agent,” and “I/O agent” are used interchangeably herein. Without being bound by theory, these agents target receptors or ligands, in the patient immune system or presented by a tumor, that are involved in the immune response to the tumor, and thereby interfere with the natural mechanisms involved in immunomodulation. The interference, generally caused by the agent binding to such a receptor or ligand, may activate, stimulate, suppress, or inhibit a natural immune response that would otherwise occur in a patient. Immune-oncology agents may be either a small molecule drug or, as is more common recently, an antibody.

The immune-oncology agents may target receptors or ligands that appear on T-cells, macrophages, natural killer cells, dendritic cells, or other antigen-presented cells, or tumor cells. Some receptors or ligands may appear in more than one cell type, and thus any reference to a receptor or ligands as appearing on a particular cell type is for convenience and is not intended to limit the scope of the disclosure or the invention.

The immune-oncology agents suitable for use with Zn(II) agents in combination therapies for treating tumors (e.g., cancer) include, but are not limited to the following agents, described below for convenience in terms of the immune component being targeted. Addition information regarding the current state of development of UO agents is provided in Burugu, S. et al., Emerging Targets in Cancer Immunotherapy, SEMINARS IN CANCER BIOLOGY 2018, 52, 39-52.

In certain embodiments, the immune-oncology agent is a programmed cell death protein 1 (PD-1) inhibitor. Information about PD-1 inhibitors, including their composition, method of preparation, formulation, dosing, and administration are as described and known to the public, including for drugs such as, e.g., nivolumab, pembrolizumab, MEDI0680 (formerly AMP-514), AMP-224, or BGB-A317.

In certain embodiments the immune-oncology agents is a programmed cell death protein ligand 1 (PD-L1) inhibitor. Information about PD-L1 inhibitors, including their composition, method of preparation, formulation, dosing, and administration are as described and known to the public, including for drugs such as, e.g., atezolizumab, avelumab, durvalumab, BMS-936559, CK-301, ZKAB001, and faz053.

In certain embodiments the immune-oncology agents is a cytotoxix T lymphocyte associated protein 4 (CTLA-4) inhibitor. Information about CTLA-4 inhibitors, including their composition, method of preparation, formulation, dosing, and administration are as described and known to the public, including for drugs such as, e.g., ipillimumab.

PD-1, PD-L1, and CTLA-4 inhibitors, as the first set of immune checkpoint inhibitors are often referred to as such, though this label should be viewed as limiting or defining the group. Many other immune checkpoints are under development as targets for inhibitors or stimulators, as discussed below. Nonetheless, the first group of approved products are important to the understanding of one of ordinary skill in the art. Additional background on the status of these I/O agents is provided in Chae, Y. K. et al., Current landscape and future of dual anti-CTLA-4 and PD-1/PD-L1 blockage immunotherapy in cancer, JOURNAL FOR IMMUNOTHERAPY OF CANCER 2018, 6, 39.

In certain embodiments the immune-oncology agents is a lymphocyte activation gene 3 (LAG-3) inhibitor. LAG-3 (CD223) is an inhibitory receptor expressed on activated T cells, B cells, and dendritic cells. It is reported that up-regulation is required to control overt activation and prevent autoimmunity, but that persistent antigen exposure in a tumor microenvironment leads to an exhausted phenotype with reduced capacity. I/O agents currently under development include REGN3767, IMP321, BMS-986016, LAG525, and MK-4280-001. Discussion of the receptor and clinical trials is provided in Andrews, L. P. et al., LAG3 (CD223) as a Cancer Immunotherapy Target, IMMUNOL, REV. 2017, 276(1), 80-96.

In certain embodiments the immune-oncology agents is a T-cell immunoglobulin- and mucin-domain-containing molecule 3 (TIM-3) inhibitor. TIM-3 is a co-inhibitory immune receptor. Its expression is reported to increase after T-cell activation, and generally marks the most dysfunctional populations of T-cells in the tumor microenvironment. I/O agents currently under development include TSR-022, LY3321367, Sym023, and MBG453.

In certain embodiments the immune-oncology agents is a T-cell immunoglobulin and ITIM domain (TIGIT) inhibitor, TIGIT is expressed on Tregs, activated CD4+ and CD8+ T cells, and NK cells. It has been reported that blocking TIGIT improved the activity of CD8+ T cells in mice receiving anti-PD1 therapy. I/O agents currently under development include OMP-313M32, BMS-986207, MTIG7192A/RG6058

In certain embodiments the immune-oncology agents is a B7-H3 (CD276) inhibitor. I/O agents currently under development include MGA271, MGD009 (a dual-affinity retargeting protein)

In certain embodiments the immune-oncology agents is a V-domain containing Ig suppressor of T-cell activation (VISTA) inhibitor. I/O agents currently under development include JNJ-61610588 and CA-170 (small molecule drug). Immune checkpoint inhibition of VISTA is reported to result in activation of T cell proliferation and cytokine production.

In certain embodiments the immune-oncology agents is an inducible T-cell costimulator (ICOS) inhibitor. ICOS is a receptor in the CD28 family of B7-binding proteins, and is expressed primarily by activated T cells. Its dual role in sustaining T-cell activation and effector functions, while also participating in Treg suppressive activity makes ICOS/ICOS-L a suitable target for immune-oncology therapy. I/O agents currently under development include JTX-2011, BMS-986226, MEDI-570, and GSK3359609, which comprise both agonist and antagonist antibodies.

In certain embodiments the immune-oncology agents is a CD27 inhibitor. CD27 is a co-stimulatory molecule on T cells that induces intracellular signals that mediate cellular activation, proliferation, effector function, and cell survival through binding to its ligand, CD70. It is reported that stimulation results in T-cell activation and antitumor activity. I/O agents under development include CDX-1127 (Varlilumab), an agonist of CD27. I/O agents targeting its ligand, CD70, include ARGX-110, BMS-936561, and vorsetuzumab mafodotin.

In certain embodiments, the immune-oncology agent is a glucocorticoid-induced TNF receptor (GITR) inhibitor, GITR is constitutively expressed on Tregs and induced on activated CD8+ and CD4+ T cells. Binding to its ligand (GITR-L) stimulates effector T cells. I/O agents under development include TRX518, MEDI1873, GWN323, and INCAGN01876. Additional background is provided in Knee, D. A. et al., Rationale for anti-GITR cancer immunotherapy, EURO. J. CANCER 2016, 67, 1-10.

In certain embodiments the immune-oncology agents is a CD47 inhibitor. Overexpression of CD47 has been observed in most cancers, and is thought to be a reason for the escape from immune surveillance by malignant cells. I/O agents under development include Hu5F9-G4, CC-90002, and TTI-621. Additional background is provided in Huang, Y. et al., Targeting CD47: the achievements and concerns of current studies on cancer immunotherapy, J. THORAC, DISEASE 2017, 9(2), E168-E174.

In certain embodiments the immune-oncology agents is a indoleamine-2,3-dioxygenase (IDO) inhibitor. IDO is an immunomodulatory enzyme that metabolizes tryptophan, which creates an immunosuppressive tumor microenvironment. Inhibition of IDO supports proliferation and activation of immune cells and offsets the immunosuppressive effects of tryptophan metabolites. UO agents under development include BMS-986205 and epacadostat, Pf-06840003, GDC-0919, and NLG802 (small molecules).

In certain embodiments the immune-oncology agents is a killer immunoglobulin-like receptor (KR) inhibitor. Blocking this receptor leads to activation of NK cells, and ultimately tumor cell destruction. I/O agents under development include IPH2101, IPH4102, and lirilumab.

In certain embodiments, the immune-oncology agent is a CD94/NKG2A inhibitor. CD94/NKG2A is an inhibitory receptor that binds HLA-E. This ligand is usually up-regulated and expressed on tumor cells, activation, thus inhibitor I/O agents that block this binding in tumor microenvironments permit NK and cytotoxic T cell responses to occur. I/O agents under development include monalizumab (IPH2201).

Kits

The invention further contemplates kits comprising a Zn(II) agent and in some embodiments, and I/O agent, such as an immune checkpoint inhibitor, an anti-PD-1 antibody, or any other of the immune-oncology agents disclosed herein for performing the disclosed methods of treatment.

Kits typically include a label indicating the intended use of the contents of the kit and instructions for use. A label includes any writing, or recorded material supplied on or with the kit, or which otherwise accompanies the kit. Accordingly, this disclosure provides a kit for treating a subject afflicted with a cancer, the kit comprising: (a) an effective dose of a Zn(II) agent and (b) instructions for using the Zn(II) agent.

In other embodiments this disclosure provides a kit for treating a subject afflicted with a cancer with a combination therapy, the kit comprising: (a) an effective dose of a Zn(II) agent and an effective dose of an immune-oncology agent; and (b) instructions for using the Zn(II) agent in combination with an immune-oncology agent according to the methods disclosed herein.

In other embodiments this disclosure provides a kit for treating a subject afflicted with a cancer with a combination therapy, the kit comprising: (a) an effective dose of a Zn(II) agent and effective doses of two immune-oncology agents; and (b) instructions for using the Zn(II) agent in combination with the immune-oncology agents according to the methods disclosed herein.

In other embodiments this disclosure provides a kit for treating a subject afflicted with a cancer, the kit comprising: (a) an effective dose of a Zn(II) agent and an effective dose of an anti-PD-1 antibody or antigen-binding portion thereof; and (b) instructions for using the Zn(II) agent in combination with an anti-PD-1 antibody in any of the methods disclosed herein.

In some embodiments, the Zn(II) agent and the I/O agent can be co-packaged in unit dosage form. In certain embodiments for treating human patients, the kit comprises an anti-human PD-1 antibody disclosed herein, e.g., nivolumab, pembrolizumab, MEDI0680 (formerly AMP-514), AMP-224, or BGB-A317. In certain embodiments, the kit comprises one or more of any of the I/O agents described above, as if each combination were separately included herein

The subject invention is further illustrated by the following examples.

### EXAMPLES

### Example 1: Preparation of Zn(II) Agent C004

The Zn(II) agent C004 (see FIG. 1A for structure; zinc salt of 45 kDa γ-PGA (unconjugated)) was prepared and formulated as follows. Sodium chloride, and water. Zinc sulfate heptahydrate and 45 kDa γ-PGA (polydisperse) were combined in aqueous solution containing sodium chloride and tromethamol and water was added to volume, and pH adjusted to 7.0 as necessary, wherein the concentrations of each component are 1 mg/mL zinc(II), at a molar ratio of Zn/glutamate monomer of 1:4.5, 10 mg/mL γ-PGA, and 1 mM tromethamol, 1 mM sodium chloride.

### Example 2: Preparation of Zn(II) Agent C005D

The Zn(II) agent C005D (see FIG. 1A for structure; zinc salt of 45 kDa γ-PGA conjugated to folate-PEG4-NH2 and cRGDfK-PEG4-NH2) was prepared and formulated as follows. First, folate-PEG4-NH2 was prepared by coupling Boc-NH2-PEG4-NH2 to the tail carboxyl group of folic acid via EDC coupling reaction, followed by TFA deprotection. Likewise, cRGDfK-PEG4-NH2 was prepared by coupling 2HN-PEG4-COOH to the lysine amine of cRGDfK by EDC coupling. Next, fully protonated 45 kDa γ-PGA (polydisperse) was conjugated to folate-PEG4-NH2 and cRGDfK-PEG4-NH2 at 1:3:3 ratio in a one-pot EDC coupling reaction where 100% of the folate and cRGDfK moieties were successfully bound to γ-PGA. The resulting γ-PGA conjugate was then purified by solvent exchange, and added with zinc sulfate at molar ratio of zinc:glutamate monomer=1:4.5 in saline, and adjusted to pH ˜6 to obtain the Zn(II) agent C005D.

### Example 3: Cytotoxicity of Zn(II) Agents Against HeLa Cells

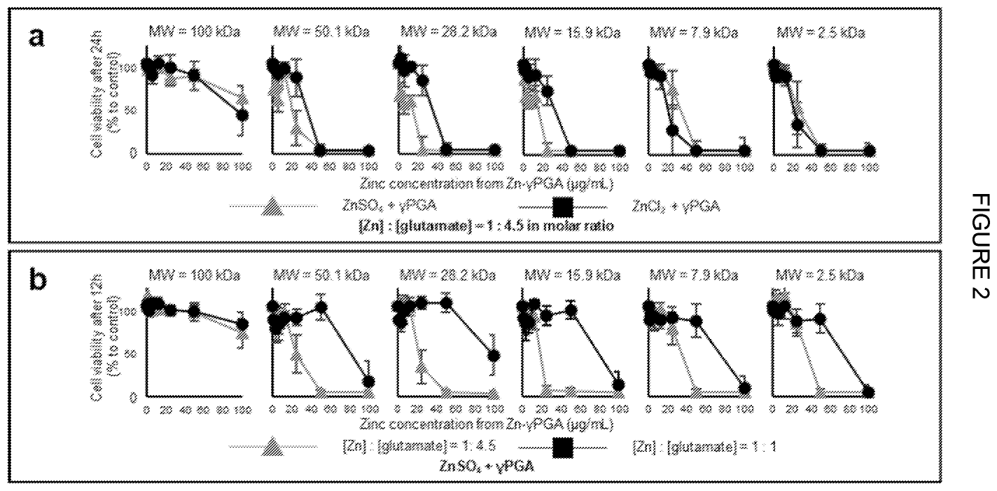

The cytotoxicity of C004 (see Example 1) and similar Zn(II) agents comprising unconjugated PGA polymers of different molecular weight against HeLa was tested as a function of zinc (II) concentration. The specific test conditions and results are shown in FIGS. 2A-2B. In FIG. 2A, the source of Zn(II) ion was zinc sulfate or zinc chloride. In FIG. 2B, the ratio of Zn:AUT was 1:4.5 or 1:1.

Cell culture preparation of HeLa cells. In accordance with ATCC guidelines, cell cultures were prepared as described by Freshney R., CULTURE OF ANIMAL CELLS (6th ed.) vol. 346 (2010). Briefly, Eagle's Minimum Essential Medium (EMEM) supplemented with 10% fetal bovine serum (FBS) was used as the complete growth medium (CGM). Fresh adherent cells grown at 37° C. and 5% CO₂ were thoroughly rinsed with 0.25% (w/v) Trypsin-EDTA (0.53 mM) solution to remove all traces of trypsin inhibitor. The cells were detached and disaggreated by adding 3.0 mL of Trypsin-EDTA solution to the rinsed flask for cell layer dispersion for 15 mins, after which 7.0 mL CGM was added an gently pipetted for further cell dissociation. The dissociated cell suspension was aliquoted at 1:4 ratio into new culture vessels and subsequently incubated at 37° C. for subculturing or for the preparation of cell line experiments.

Cell viability test. CCK-8 tests were performed in accordance with the manufacturer's instructions. Briefly, subject cultured cells were incubated with specific treatment agents for specific time in 96 well plates. At the condition of interest, 10 μL of WST-8 solution was added for 1 hr incubation at 37° C. The plates were then measured for the absorbance at 460 nm for cell viability quantification.

The studies reported in FIGS. 2A-2B showed that Zn(II) agent showed a maximum cytotoxicity in the midrange of the polymer molecular weights tested and less cytoxicity at the upper end compared to the lower end. Also, the ratio of Zn:GAU ratio of 1:4.5 was consistently more cytotoxic than the 1:1 ratio agents for all γ-PGA less than 100 kDA.

### Example 4: IC50 Determination of C004 and C005D in HeLa Cells

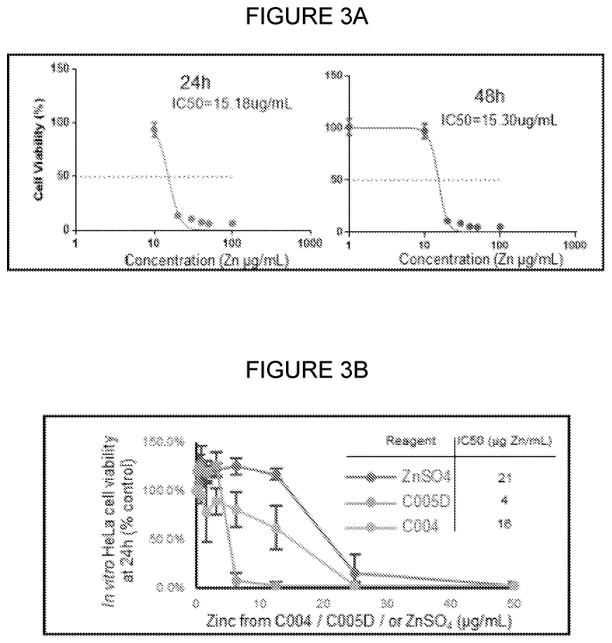

The IC50 values for C004 (see Example 1) and C005D (see Example 2) in HeLa cells were determined. The test methods described in Example 3 were used, and the results are shown in FIGS. 3A-3B.

As shown in FIG. 3A, IC50 for C004 against HeLa was 15.18 μg Zn/mL and 15.30 μg Zn/mL at 24h and 48h, respectively. As shown in FIG. 3B, the IC50 for C005D was 4 μg Zn/mL at 24h.

### Example 5: Analysis of Cell Death Mechanism

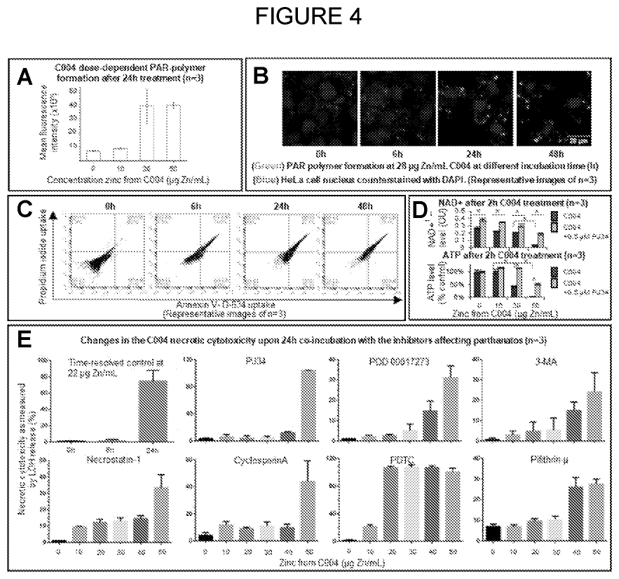

Exposing HeLa cells in vitro to C004 induced time and dose dependent accumulation of poly(ADP-ribose) polymer (PAR polymer) after 6h, which coincided with the appearance of necrotic cells showing condensed nucleus (FIGS. 4A and 4B). Flow cytometric characterization of the death mode using propidium iodide (PI) and Annexin-V-D-634 (AnxV) showed a corroborating pattern of necrotic cell death whereby the dying cells showed simultaneous uptake of both PI and AnxV after 6h incubation at 22 μg Zn/mL C004, indicating leaky nuclear and cellular membranes (FIG. 4C). Time-resolved lactate dehydrogenase (LDH) release assay, on the other hand, indicated necrotic leakage only after 24h under the same conditions (see FIG. 4E).

Consistent with events reported to be characteristic of parthanatos (see Andrabi, S. A. et al. PNAS, vol. 111, p. 10209-10214 (2014)), this cell death was preceded by the dose-dependent depletion of cellular NAD+ and ATP after 2h treatment (FIG. 4D). 24h co-incubation of C004 with a specific PARP-1 inhibitor PJ34 led to the suppression of the necrotic death as assayed by LDH release assay (FIG. 4E). Similarly, as also shown in FIG. 4E, inhibitors of some of the key downstream enzymes in parthanatos also led to the suppression of the necrotic cytotoxicity in HeLa cells, including cyclosporinA (MPTP formation), necrostatin-1 (RIP1), 3-MA (p62/SQSTM1), and pifithrin-μ (dual inhibition of p53 and p62/SQSTM1). To the contrary, agonism of p62/SQSTM1 from the JNK activation by PDTC did not cause a shift in the IC50 necrotic cytotoxicity. Lastly, specific PARG inhibitor PDD 00017273 also suppressed the necrotic cytotoxicity, which suggested PARP1-PARG NAD+ catabolism cycle as a cytotoxicity determining factor during parthanatos. These findings collectively suggested the previously proposed RIP1 and p62/SQSTM1 coordinated necroptosis as the main downstream cytotoxic mechanism of C004-induced parthanatos (FIG. 18). See Goodall, M. L. et al., Developmental Cell, vol. 37, 337-349 (2016).

### Example 6: Effect of Zn/γ-PGA Agent C004 on Cell Viability in Fifty Cancer Cell Lines

In this study the potential effect of a Zn(II) agent on cell viability was investigated in 50 cancer cell lines. The 50% inhibitory concentration (IC50) was determined in the cancer cell lines using CellTiter-Glo luminescent cell viability assay after incubation with different concentrations of active agents. Each cell line was treated with active Zn(II) agent, a standard chemotherapy drug as reference control, and culture medium as vehicle control.

Abbreviations used in this example: CTG: CellTiter-Glo; DMSO: Dimethyl Sulfoxide; FBS: Fetal Bovine Serum; IC50: 50% Inhibitory Concentration; ID: Identity; Lum: Luminescence; PBS: Phosphate Buffered Saline; RT: Room Temperature.

Cell Lines. All cell lines were cultured in media supplemented with 10-15% FBS, at a temperature of 37° C., 5% CO₂, and 95% humidity. The reference control drug for all cell lines was cisplatin, to be used for genomic analysis. The cell lines tested, the tissue of origin are shown below in Table 1:

| | Cell | Tissue |
|---|---|---|
| No. | Line Name | Origin |
| 1 | AMO-1 | Blood |
| 2 | CA46 | Blood |
| 3 | Daudi | Blood |
| 4 | EOL-1 | Blood |
| 5 | HL-60 | Blood |
| 6 | JJN-3 | Blood |
| 7 | Jurkat clone E6-1 | Blood |
| 8 | JVM-3 | Blood |
| 9 | K-562 | Blood |
| 10 | KARPAS-422 | Blood |
| 11 | Kasumi-1 | Blood |
| 12 | L-363 | Blood |
| 13 | ML-2 | Blood |
| 14 | MOLM-13 | Blood |
| 15 | MOLM-16 | Blood |
| 16 | MOLP8 | Blood |
| 17 | Molt-4 | Blood |
| 18 | MV-4-11 | Blood |
| 19 | NALM-6 | Blood |
| 20 | NAMALWA | Blood |
| 21 | NCI-H929 | Blood |
| 22 | OCI-AML2 | Blood |
| 23 | OCI-LY-19 | Blood |
| 24 | Pfeiffer | Blood |
| 25 | Raji | Blood |
| 26 | Ramos | Blood |
| 27 | SU-DHL-6 | Blood |
| 28 | THP-1 | Blood |
| 29 | U-937 | Blood |
| 30 | WSU-NHL | Blood |
| 31 | DU4475 | Breast |
| 32 | HCC1954 | Breast |
| 33 | MCF7 | Breast |
| 34 | HeLa | Cervix |
| 35 | HCT 116 | Colorectum |
| 36 | Hep G2 | Liver |
| 37 | Hep3B | Liver |
| 38 | JHH-7 | Liver |
| 39 | SK-HEP-1 | Liver |
| 40 | A549 | Lung |
| 41 | Calu-3 | Lung |
| 42 | NCI-H446 | Lung |
| 43 | NCI-H460 | Lung |
| 44 | OVCAR-3 | Ovary |
| 45 | SK-OV-3 | Ovary |
| 46 | AsPC-1 | Pancreas |
| 47 | PANC-1 | Pancreas |
| 48 | DU 145 | Prostate |
| 49 | PC-3 | Prostate |
| 50 | MES-SA/DX5 | Uterus |

Materials and Reagents.
General cell culture reagents and plastic.
FBS, (Cat #FND500, ExCell Bio. Store at −20° C.)
FBS, (Cat #10091148, Gibco. Store at −20° C.)
96-Well Flat Clear Bottom Black Polystyrene TC-Treated Microplates (Cat #3340, Corning).
   CellTiter-Glo® Luminescent Cell Viability Assay (Cat #G7572, Promega. Store at −20° C.)
   Substrate is sufficient for 1,000 assays at 100 μL per assay in 96-well plates.

### Including:

1×100 mL CellTiter-Glo® Buffer
1× vial CellTiter-Glo® Substrate (lyophilized)

Reagent Preparation
a. Thaw the CellTiter-Glo Buffer, and equilibrate to room temperature prior to use. For convenience, the CellTiter-Glo Buffer may be thawed and stored at room temperature for up to 48 h prior to use.
b. Equilibrate the lyophilized CellTiter-Glo Substrate to room temperature prior to use.
c. Transfer the appropriate volume (100 mL) of CellTiter-Glo Buffer into the amber bottle containing CellTiter-Glo Substrate to reconstitute the lyophilized enzyme/substrate mixture. This forms the CellTiter-Glo Reagent. Note: The entire liquid volume of the CellTiter-Glo Buffer bottle may be added to the CellTiter-Glo Substrate vial.
d. Mix by gently vortexing, swirling or by inverting the contents to obtain a homogeneous solution. The CellTiter-Glo Substrate should go into solution easily in less than one minute.

Zn(II) Agent and Reference Control Drug.

The Zn(II) agent test article was:

| Item | | Sample | | Storage |
|---|---|---|---|---|
| No. | Zn(II) Agent | Description | Volume (mL) | Conditions |
| 1 | C004 | Liquid | 10 | 4° C. |

The stock solution of compounds was divided into aliquots and these were stored in 4° C. freezer. The zinc(II) agent C004 composition was:

| No. | Substance | % | Per mL | |
|---|---|---|---|---|
| 1. | Zinc sulfate heptahydrate | 0.440 | 4.40 | mg |
| | equivalent to 1 mg Zinc | | | |
| 2. | PGA powder with MW | 2.831 | 28.31 | mg |
| | 20-45 kDa | | | |
| 3. | Sodium chloride | 0.650 | 6.50 | mg |
| 4. | Tromethamol | 0.070 | 0.70 | mg |
| 5. | Water for injection | Ad | Ad 1 | mL |
| | | 100 % | | |

The reference control drug was:

| | Molecular | | | | | | Expire |
|---|---|---|---|---|---|---|---|
| Subject | Weight | Lot# | Package | Supplier | Properties | Storage | Date |
| Cisplatin | 300.05 | D061881 | 50 mg | Hospira | Liquid | RT | 2018 Sep. 13 |
| | | AA | in | Australia Pty | | | |
| | | | 50 mL | Ltd | | | |

Equipment. EnVision Multi Label Reader 2104-0010A, Perkin Elmer (USA), Equip ID: TAREA0020; Countstar, Inno-Alliance Biotech (USA), Equip ID: BEANA0020; Forma Series II Water Jacket CO2 Incubator, Thermo Scientific (USA), Equip ID: BEINC0190/BEINC0200/BEINC0220/BEINC0260; Biological safety Cabinet, Thermo Scientific (USA), Equip ID: BEBSC0170/BEBSC0180/BEBSC0250/BEBSC0270; Clean bench, HDL Apparatus (China), Equip ID: BACLB0390; Biomek FXP Laboratory Automation Workstation, BECKMAN COULTER (USA), Equip ID: BESTA0010; Inverted Microscope, Olympus CKX41SF (Japan), Equip ID: BEMIC0190; Multidrop combi, Thermo Scientific (USA), Equip ID: BEPFL0010.

Procedure for Determining Half Maximal Inhibition Concentration IC50
1. Harvested cells during the logarithmic growth period and count cell number using Count-star.
2. Adjusted cell concentrations to 4.44×10⁴ cells/mL with respective culture medium.
3. Added 90 μL cell suspensions to two 96-well plates (plates A and B) with the final cell density of 4×10³ cells/well. (cell concentration was adjusted according to the data base or density optimization assay.)
4a. Next day: For the plates of TO reading:
   1) Added 10 μL culture medium to each well of plate A for TO reading.
   2) Equilibrated the plate and its content at RT for approximately 30 min.
   3) Added 50 μL CellTiter-Glo reagent to each well.
   4) Mixed content for 5 min on an orbital shaker to induce cell lysis.
   5) Allowed the plate to incubate at RT for 20 min to stabilize luminescent signal.
   6) Recorded luminescence (TO) using EnVision Multi Label Reader.
4b. For the plates of test reading:
   1) Prepared 10× solution of Zn(II) agent test article. Top working concentration: 50 μg/mL of test article in media with 2/2.5-fold serial dilutions to achieve 9 dose levels of 50, 25, 10, 5, 2.5, 1, 0.5, 0.25, and 0 μg/mL concentration of the Zn(II) agent.
   2) Prepared 10× reference control solutions Cisplatin. Top working concentration: 100 μM in media with 3.16-fold serial dilutions to achieve 9 dose levels of 100 μM, 31.6 μM, 10 μM, 3.16 μM, 1 μM, 316 nM, 100 nM, 31.6 nM, and 10 nM concentration of cisplatin.
   3) Dispensed 10 μL (10×) drug solution of both test article and reference control in each well (triplicate for each drug concentration) of the plate B.
   4) Incubated the test plate B for 96 h in the humidified incubator at 37° C. with 5% CO₂, and then measured by means of CTG assay.
   5) Equilibrated the plate and its content at RT for approximately 30 min.
   6) Added 50 μL CellTiter-Glo reagent to each well.
   7) Mixed contents for 5 min on an orbital shaker to induce cell lysis.
   8) Allowed the plates to incubate at RT for 20 min to stabilize luminescent signal.
   9) Recorded luminescence.

Data Analysis
10) The data was displayed graphically using GraphPad Prism 5.0.
11) To calculate absolute IC50 (EC50), a dose-response curve was fitted using nonlinear regression model with a sigmoidal dose response. The formula for calculating surviving rate is shown below and the absolute IC50 (EC50) was calculated according to the dose-response curve generated by GraphPad Prism 5.0.
12) The surviving rate (%)=(LumTest article−LumMedium control)/(LumNon-treated−LumMedium control)×100%.

Results.

TABLE 2-1
Summary of Absolute IC50s & Maximal Inhibition in 29 Cell Lines

| | | Absolute IC50 | | % inhibition at | | | | | |
|---|---|---|---|---|---|---|---|---|---|
| Cell | | (μg/mL) | | top conc. | | HillSlope | | R² | |
| No. | Cell lines | Zepitide | Cisplatin | Zepitide | Cisplatin | Zepitide | Cisplatin | Zepitide | Cisplatin |
| 1 | A549 | 18.99 | 0.88 | 99.95% | 97.57% | −3.87 | −0.93 | 0.967 | 0.985 |
| 2 | NCI-H446 | 3.72 | 0.35 | 99.99% | 99.99% | −8.00 | −0.65 | 0.996 | 0.982 |
| 3 | NCI-H460 | 12.60 | 0.15 | 99.97% | 99.98% | −6.11 | −1.63 | 0.992 | 0.998 |
| 4 | MES-SA/DX5 | 30.40 | 0.56 | 98.55% | 99.97% | −2.41 | −1.75 | 0.993 | 0.998 |
| 5 | DU 145 | 9.99 | 0.20 | 99.97% | 99.24% | −2.77 | −2.37 | 0.979 | 0.992 |
| 6 | SK-OV-3 | 19.56 | 0.54 | 99.90% | 98.22% | −3.01 | −1.68 | 0.983 | 0.996 |
| 7 | KARPAS- | 3.08 | 0.37 | 99.99% | 99.99% | −11.41 | −1.04 | 0.996 | 0.992 |
| | 422 | | | | | | | | |
| 8 | ML-2 | 3.30 | 0.32 | 99.99% | 100.00% | −8.18 | −3.28 | 0.999 | 0.998 |
| 9 | SK-HEP-1 | 13.26 | 2.13 | 99.96% | 99.93% | −8.13 | −1.28 | 0.998 | 0.993 |
| 10 | PC-3 | 14.96 | 1.13 | 99.95% | 93.27% | −10.83 | −1.55 | 1.000 | 0.997 |
| 11 | HL-60 | 8.61 | 0.29 | 99.98% | 99.99% | −2.29 | −2.08 | 0.986 | 0.994 |
| 12 | Jurkat clone | 9.40 | 0.17 | 99.98% | 99.99% | −3.20 | −2.37 | 0.999 | 0.998 |
| | E6-1 | | | | | | | | |
| 13 | JVM-3 | 5.67 | 0.14 | 99.99% | 99.99% | −6.18 | −1.70 | 0.994 | 0.995 |
| 14 | HCC1954 | 12.94 | 1.59 | 99.85% | 99.87% | −6.38 | −1.98 | 0.991 | 0.997 |
| 15 | Hep G2 | 17.32 | 0.55 | 99.94% | 98.96% | −8.33 | −0.90 | 0.988 | 0.991 |
| 16 | OVCAR-3 | 19.46 | 1.08 | 99.91% | 97.70% | ~−20.23 | −2.96 | 0.997 | 0.990 |
| 17 | DU4475 | 19.44 | 0.31 | 99.95% | 99.94% | ~−25.59 | −2.54 | 0.999 | 0.999 |
| 18 | MCF7 | 16.75 | 1.64 | 99.84% | 96.37% | −5.91 | −0.60 | 0.992 | 0.985 |
| 19 | HeLa | 12.96 | 0.05 | 99.97% | 99.91% | −5.58 | −1.70 | 0.983 | 0.998 |
| 20 | MOLM-16 | 14.31 | 0.15 | 99.93% | 99.96% | −6.38 | −1.65 | 0.993 | 0.996 |
| 21 | Daudi | 2.68 | 0.11 | 99.99% | 99.86% | −8.00 | −1.59 | 0.981 | 0.995 |
| 22 | L-363 | 19.63 | 0.83 | 99.93% | 99.98% | ~−18.33 | −2.08 | 0.987 | 0.992 |
| 23 | K-562 | 13.11 | 1.02 | 99.96% | 97.71% | −8.24 | −1.81 | 0.997 | 0.986 |
| 24 | HCT-116 | 24.10 | 2.89 | 97.81% | 91.19% | −3.75 | −2.03 | 0.998 | 0.987 |
| 25 | CA46 | 13.41 | 0.21 | 99.96% | 99.92% | −8.75 | −1.96 | 0.988 | 0.996 |
| 26 | Calu-3 | 18.99 | 0.30 | 99.19% | 82.29% | −1.13 | −1.15 | 0.986 | 0.992 |
| 27 | JHH-7 | 14.20 | 0.44 | 99.96% | 99.16% | −6.77 | −0.80 | 0.998 | 0.986 |
| 28 | Kasumi-1 | 23.07 | 0.71 | 99.85% | 99.98% | ~−18.03 | −1.18 | 0.989 | 0.989 |
| 29 | PANC-1 | 15.74 | 1.08 | 99.96% | 96.35% | −10.07 | −0.62 | 0.996 | 0.982 |

TABLE 2-2
Summary of Absolute IC50s & Maximal Inhibition in 13 Cell Lines

| | | Absolute IC50 | | % inhibition at | | | | | |
|---|---|---|---|---|---|---|---|---|---|
| Cell | | (μg/mL) | | top conc. | | HillSlope | | R² | |
| No. | Cell lines | Zepitide | Cisplatin | Zepitide | Cisplatin | Zepitide | Cisplatin | Zepitide | Cisplatin |
| 30 | NAMALWA | 3.04 | 0.07 | 99.99% | 99.99% | −9.19 | −3.97 | 0.995 | 0.996 |
| 31 | Raji | 3.04 | 0.50 | 99.98% | 99.57% | −10.67 | −2.98 | 0.993 | 0.997 |
| 32 | OCI-LY-19 | 1.87 | 0.04 | 100.00% | 99.99% | −2.40 | −2.61 | 0.989 | 0.997 |
| 33 | MV-4-11 | 5.45 | 0.09 | 99.99% | 99.99% | ~−17.53 | −2.06 | 0.994 | 0.993 |
| 34 | WSU-NHL | 4.37 | 0.06 | 99.99% | 99.99% | −3.64 | −1.77 | 0.994 | 0.996 |
| 35 | Molt-4 | 5.70 | 0.10 | 99.99% | 99.99% | −11.32 | −2.01 | 0.997 | 0.997 |
| 36 | NALM-6 | 9.78 | 0.21 | 99.97% | 99.64% | −6.08 | −2.22 | 0.982 | 0.998 |
| 37 | NCI-H929 | 5.32 | 0.64 | 99.92% | 99.98% | −5.00 | −1.83 | 0.993 | 0.994 |
| 38 | U-937 | 8.26 | 0.37 | 99.98% | 99.99% | ~−33.47 | −4.57 | 0.993 | 0.996 |
| 39 | MOLP8 | 13.02 | 0.16 | 99.66% | 99.98% | −3.73 | −2.16 | 0.991 | 0.996 |
| 40 | Pfeiffer | 5.77 | 0.83 | 99.96% | 99.98% | −8.73 | −1.79 | 0.996 | 0.996 |
| 41 | SU-DHL-6 | 11.46 | 0.38 | 99.97% | 99.99% | −4.35 | −1.11 | 0.996 | 0.995 |
| 42 | OCI-AML2 | 48.08 | 0.33 | 99.85% | 99.96% | ~−21.77 | −2.66 | 0.982 | 0.994 |

TABLE 2-3
Summary of Absolute IC50s & Maximal Inhibition in 8 Cell Lines

| | | Absolute IC50 | | % inhibition at | | | | | |
|---|---|---|---|---|---|---|---|---|---|
| Cell | | (μg/mL) | | top conc. | | HillSlope | | R² | |
| No. | Cell lines | Zepitide | Cisplatin | Zepitide | Cisplatin | Zepitide | Cisplatin | Zepitide | Cisplatin |
| 43 | JJN-3 | 12.93 | 0.28 | 99.96% | 99.81% | −9.20 | −1.17 | 0.999 | 0.991 |
| 44 | MOLM-13 | 15.57 | 0.16 | 99.96% | 99.98% | −12.04 | −2.08 | 0.994 | 1.000 |
| 45 | AMO-1 | 13.46 | 0.27 | 99.96% | 99.99% | −7.04 | −3.34 | 0.989 | 0.998 |
| 46 | EOL-1 | 6.68 | 0.16 | 99.98% | 99.99% | −2.54 | −1.87 | 0.988 | 0.995 |
| 47 | THP-1 | 12.53 | 0.39 | 99.95% | 99.98% | −5.31 | −3.03 | 0.989 | 0.999 |
| 48 | AsPC-1 | 8.67 | 0.67 | 99.97% | 81.58% | −2.32 | −1.47 | 0.991 | 0.987 |
| 49 | Ramos | 3.04 | 0.17 | 99.99% | 99.99% | −13.02 | −2.13 | 0.998 | 0.993 |
| 50 | Hep3B | 7.69 | 0.78 | 99.97% | 99.58% | −7.31 | −1.50 | 0.993 | 0.988 |

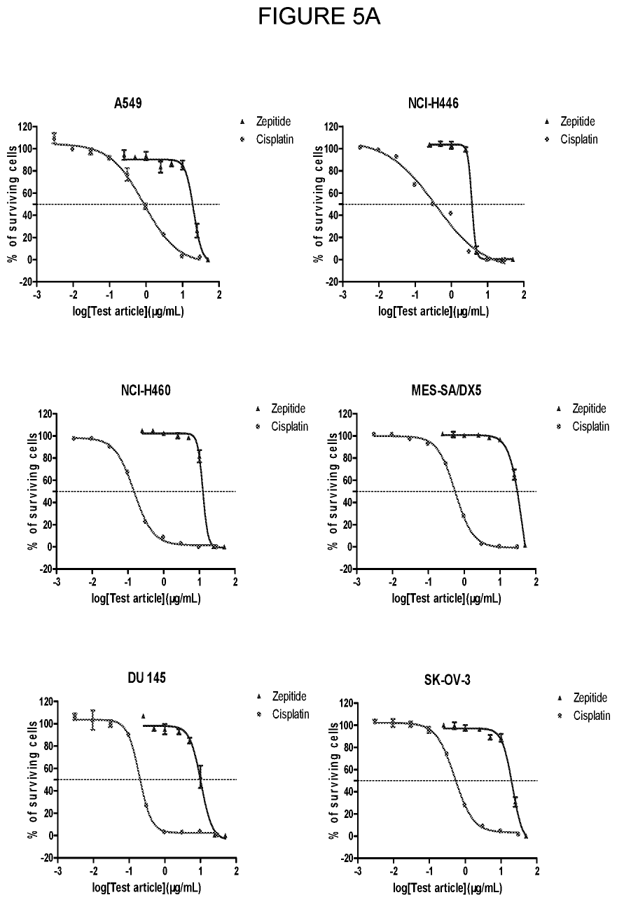

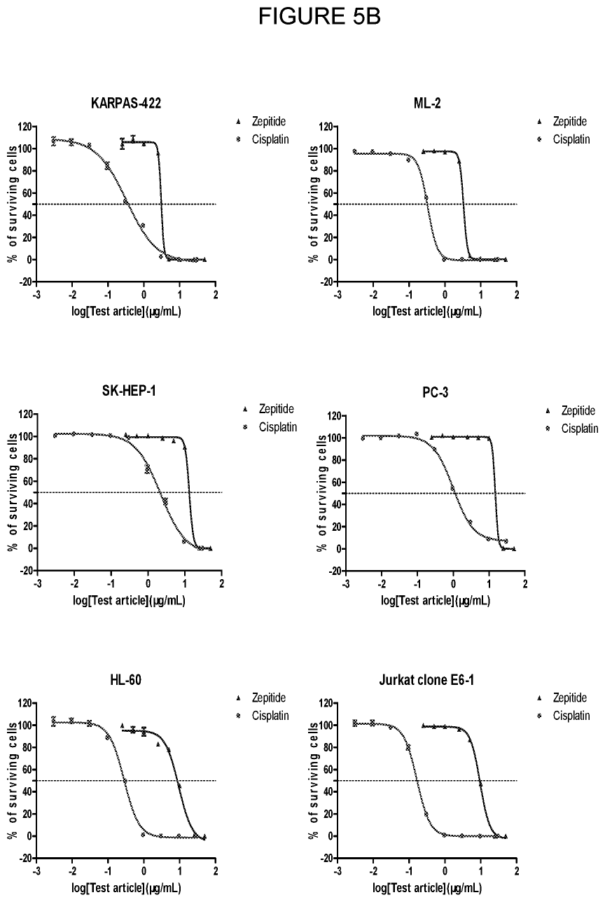

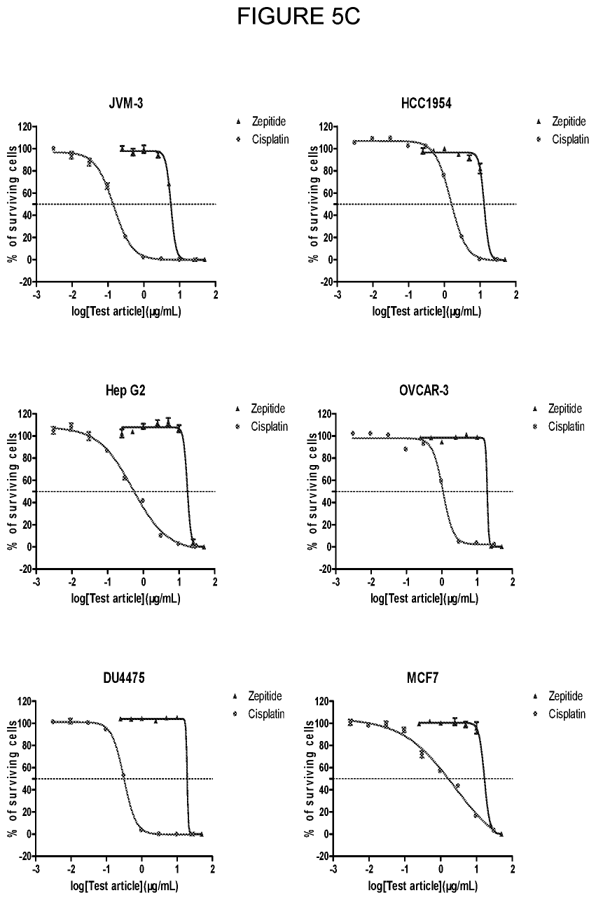

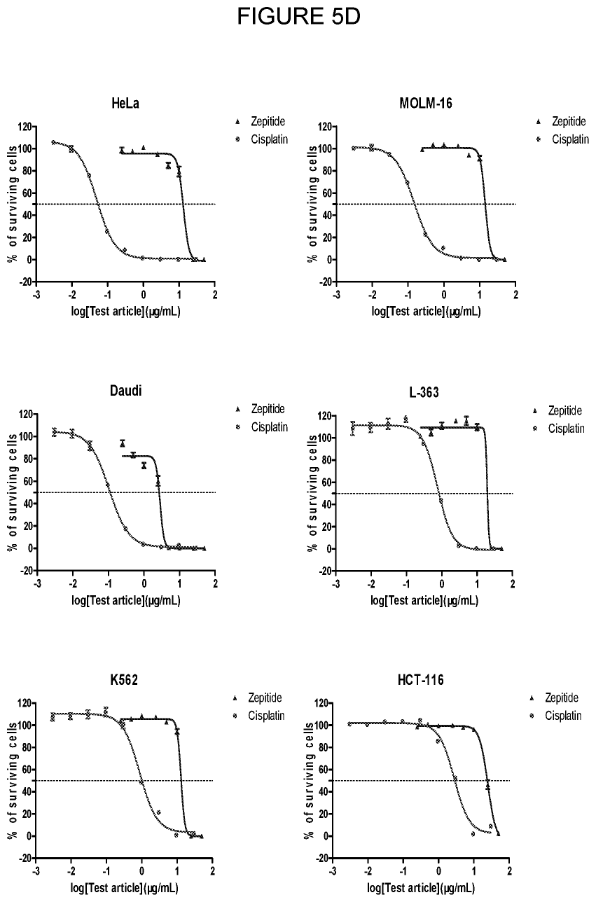

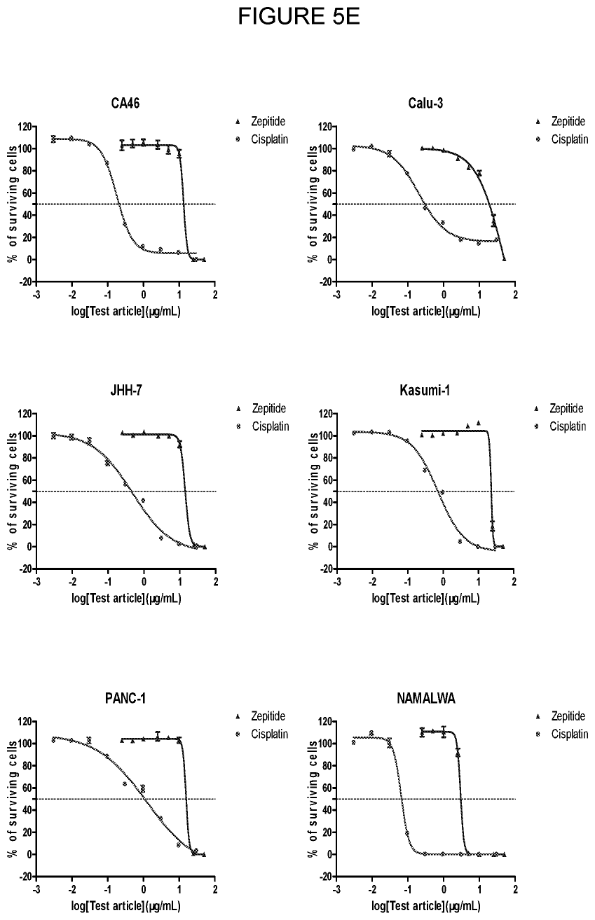

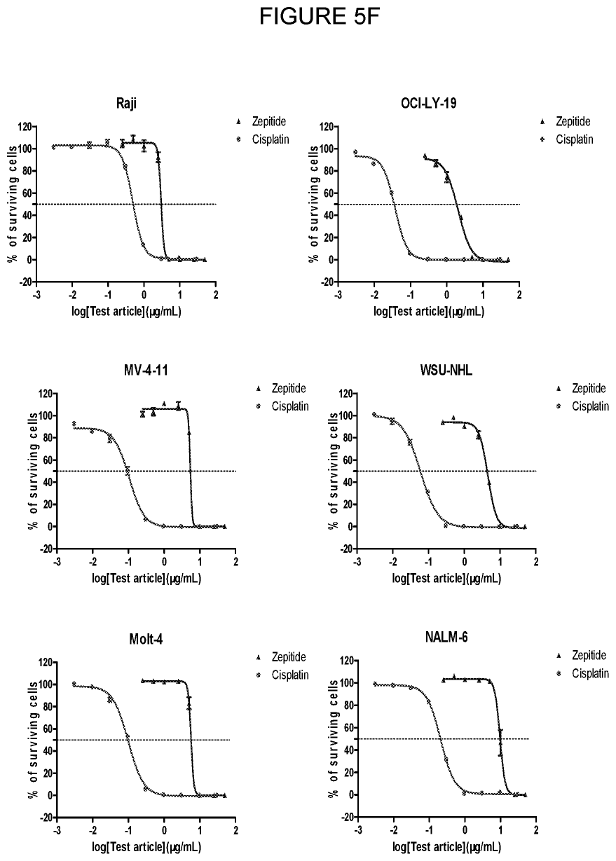

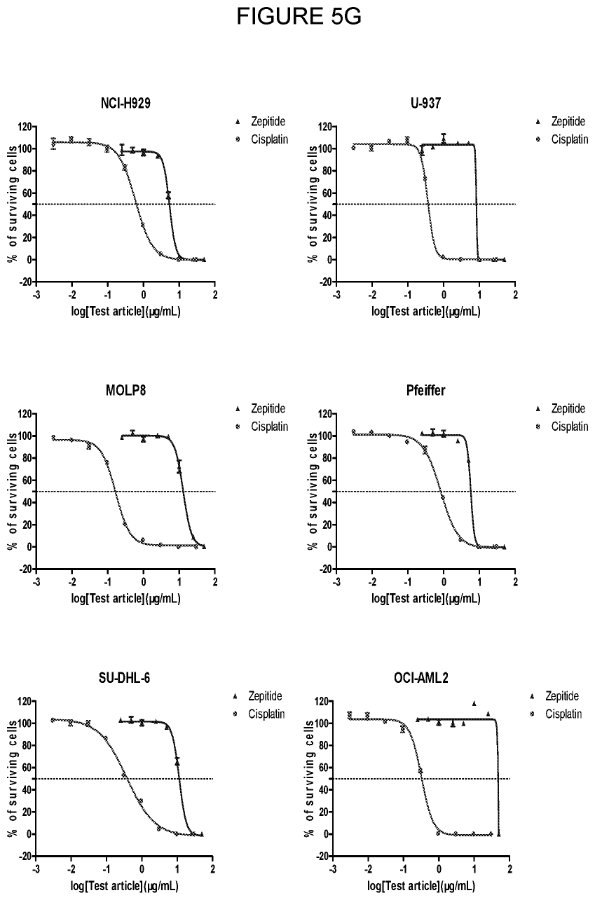

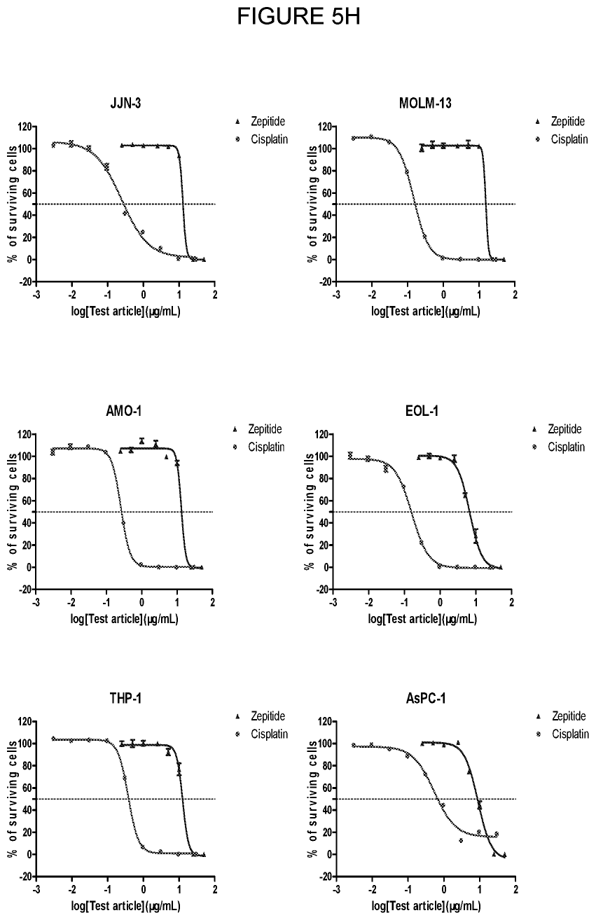

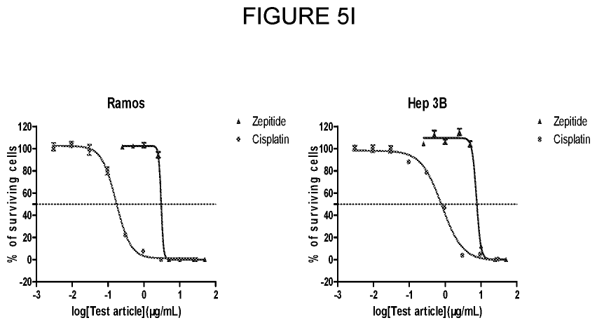

The dose response curves for each cell line is shown in FIGS. 5A-5I.

### Example 7: Genetic Instability Mutation (GIM) Analysis

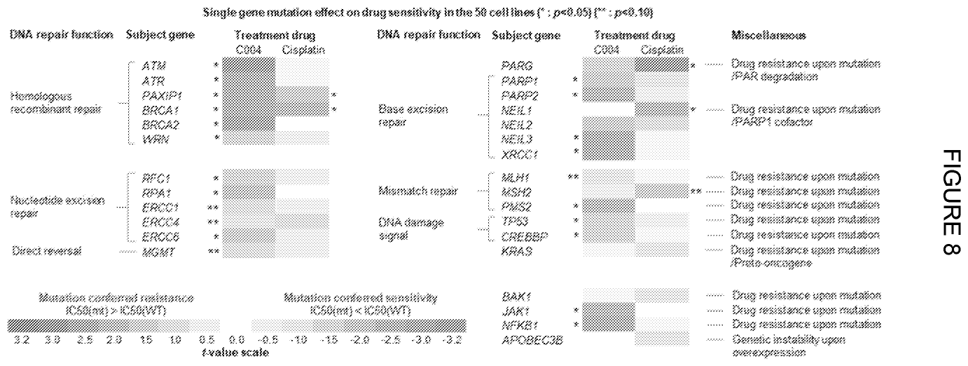

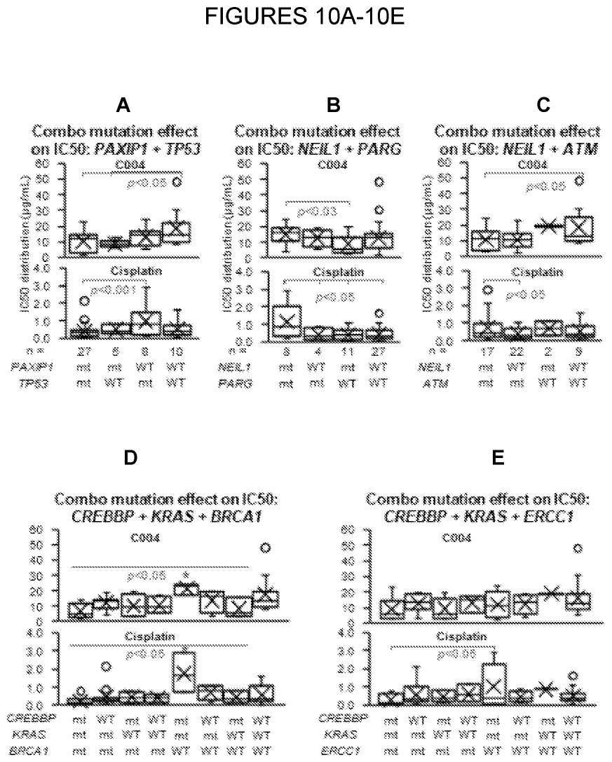

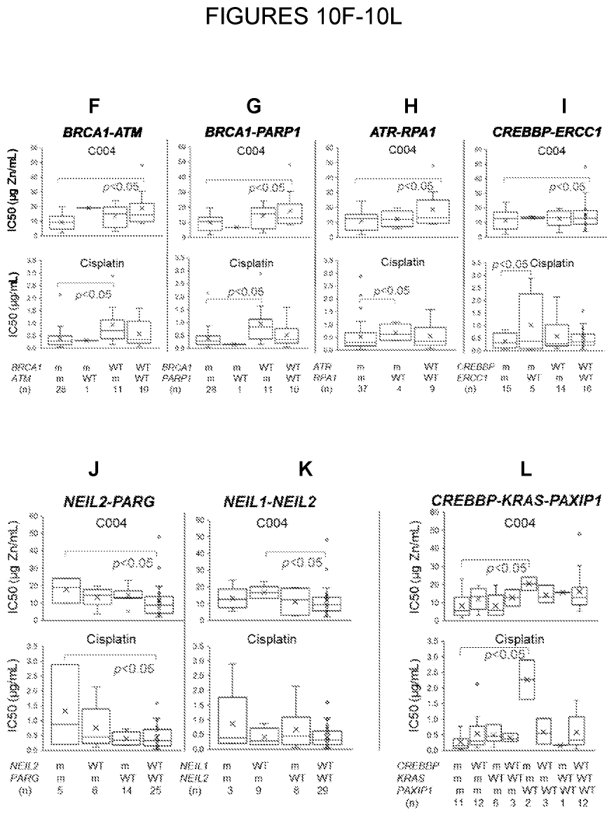

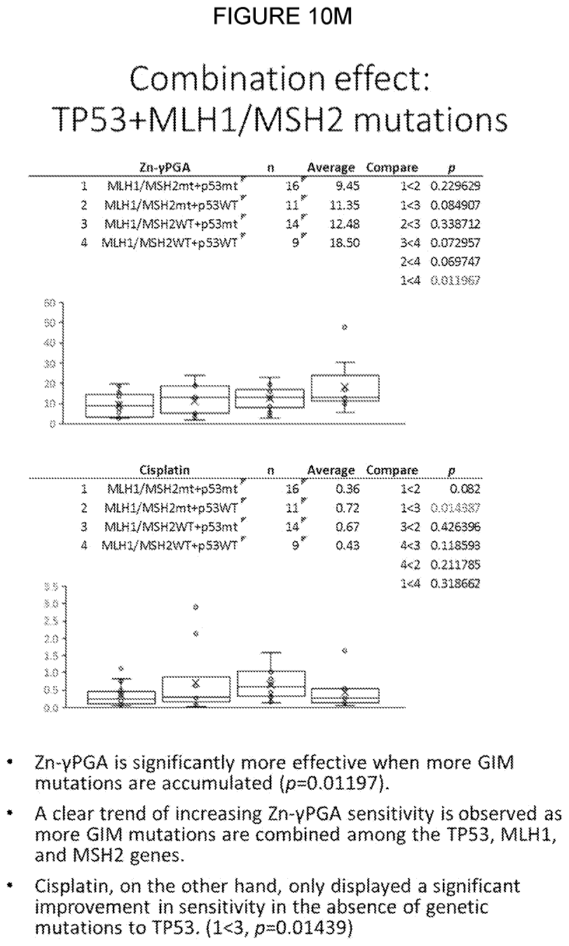

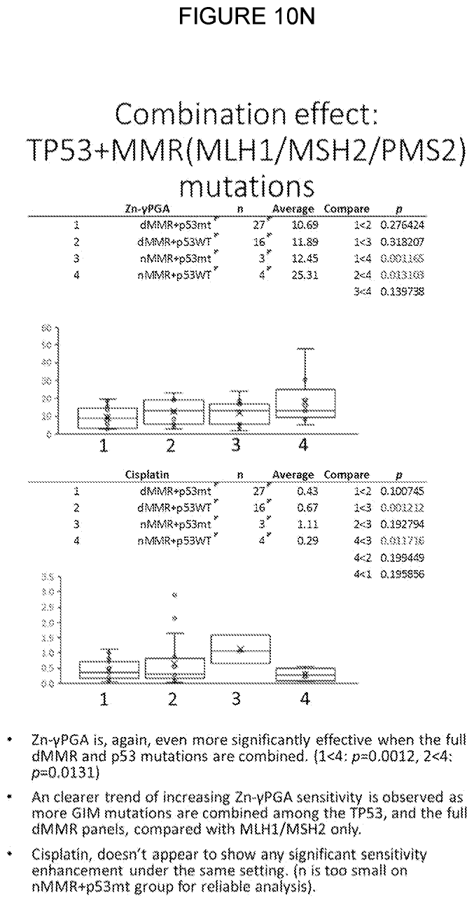

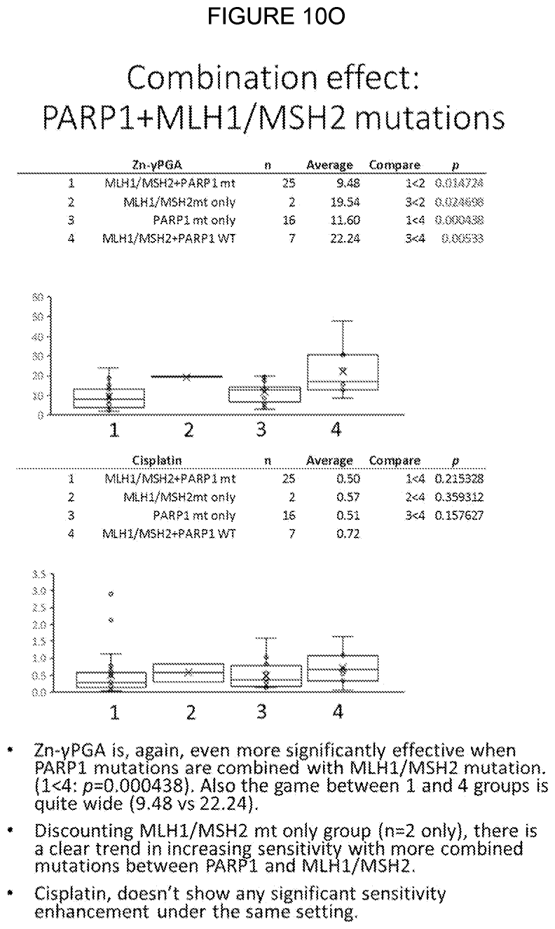

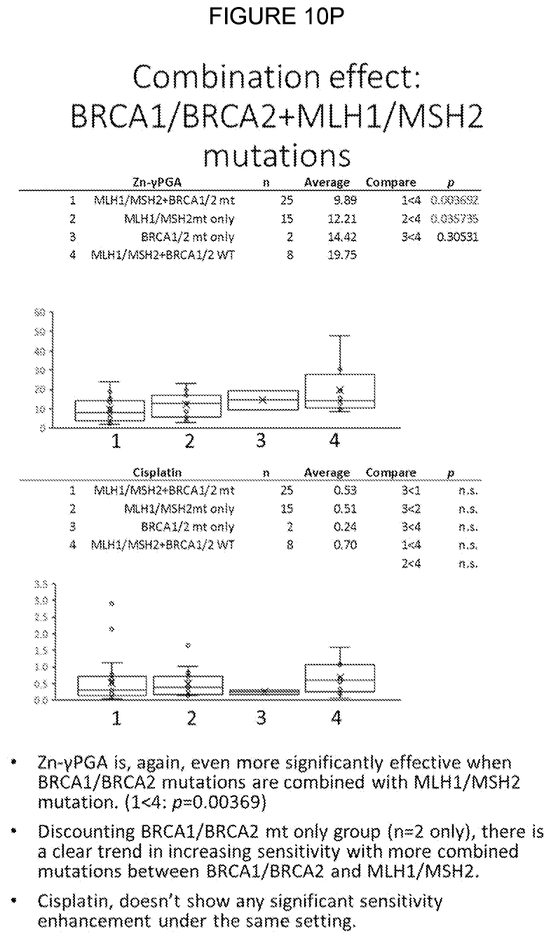

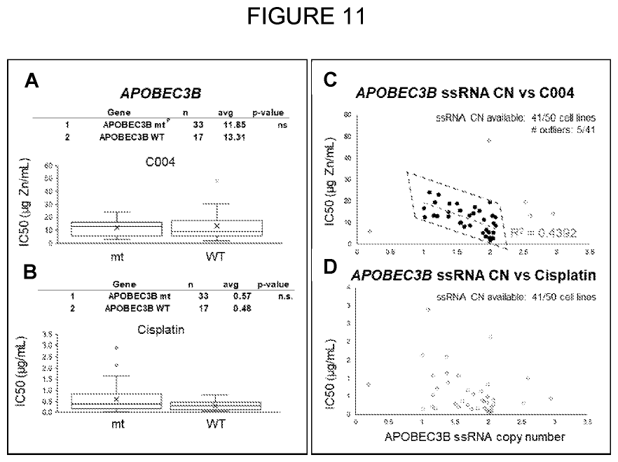

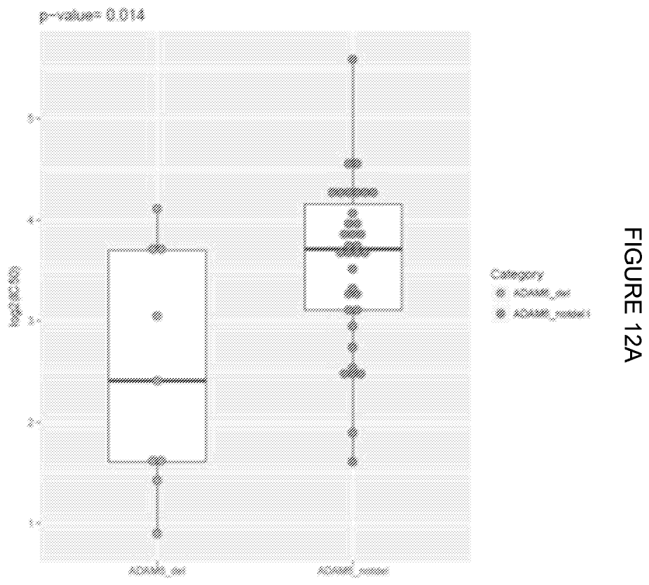

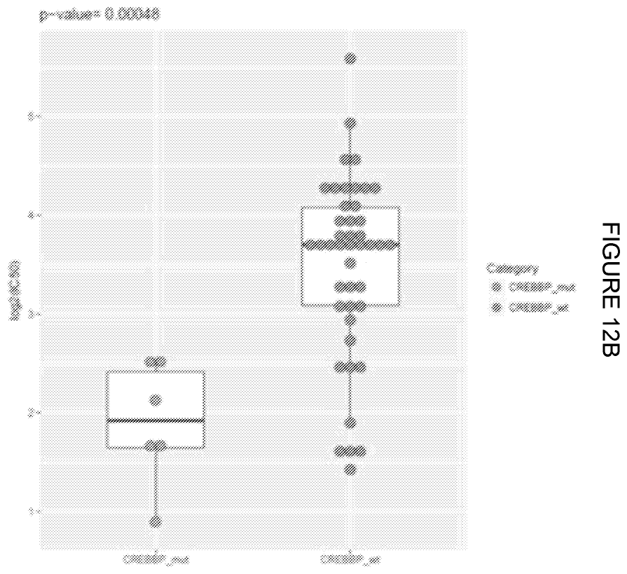

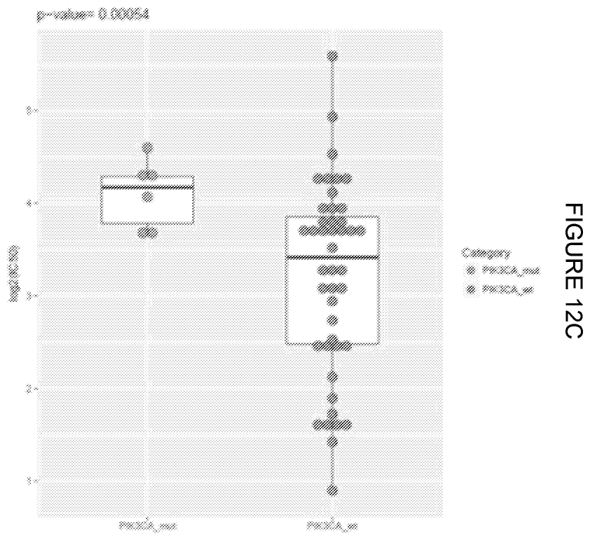

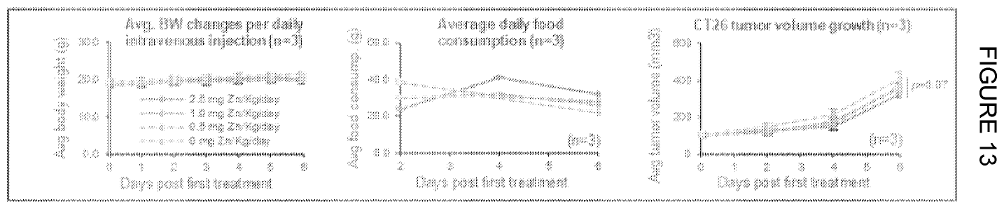

Given C004's parthanatos mechanism, genetic instability mutations should heighten cancer sensitivity to C004 by conferring greater PARylation potential. Hence, single mutation effect analyses surrounding homologous recombinant repair, nucleotide excision repair, direct reversal, base excision repair, mismatch repair, DNA damage signal, and other related functions were tested using the screening results. The cisplatin data was used as the positive control for ensuring the quality of the genomic information gathered on the cell lines and meaningful interpretation of their biological significance, which included the association of the cisplatin resistance to MSH2mt, sensitivity to BRCA1mt (see FIG. 8), apoptosis resistance conferred by PARGmt, many synthetic lethality examples between the combinations of most DNA-repair genes such as ATM and BRCA1 (FIG. 10F), and the resistance to CREBBmt+KRASmt with BRCA1WT (FIG. 10D). Application of the same genomic information against C004 IC50 distribution demonstrated drug sensitivity association to the mutation of most DNA-repair genes tested including PARP1, PARP2, TP53, MGMT, XRCC1, ERCC1, ERCC4, ERCC6, RFC1, MLH1, PMS2, ATM, ATR, BRCA1, BRCA2, PAXIP1, and WRN. JAK1mt, CREBBPmt, NEIL3mt, and NFKB1mt were also associated with increased sensitivity (FIG. 8 and FIGS. 10F-10L). Further testing on the hypothesis of enhanced C004 efficacy against mutational load by studying APOBEC3B overexpression effect, one of the most prevalent driver of mutagenesis, also showed a consistent result by showing a negative correlation between its ssRNA copy number and C004 IC50 value distribution (FIGS. 11A-11D). Together all these observations support the determination that the cytotoxicity of C004 involves the parthanatos mechanism.

Further in support of this mechanism, mutations in the essential partners for efficient PARP1's activity toward NAD+ depletion such as PARG (PAPR1-PARG NAD+ catabolic cycle), NEIL1 or NEIL2 (initiation of base-excision repair) were observed with weak but consistent tolerance (FIG. 8), while their combinations mounted to significant attenuation of C004 efficacy (FIGS. 10B-10C and FIGS. 10J-10K). The CREBBPmt-KRASmt-BRCA1WT combination that showed strong resistance against cisplatin was also associated with lesser but significant resistance to C004, noting the mutation combo as a potential resistance mechanism. Lastly, inspection of the known mutational drivers for the development of resistance to PD-L1 checkpoint inhibitors (JAK1 24), apoptosis (TP53, BAK1), and various antineoplastic drugs and multidrug resistance (TP53 25, 26, MLH1, MSH2, PMS2 27 and NFKB1) showed that C004 cytotoxicity is indifferent (BAK1, MSH2) if not more active (JAK1, TP53, MLH1, PMS2, NFKB1) toward these drug resistance driver mutations (FIG. 8, FIG. 10).

Following the 50 cell line screening tests (OmniScreen™) described in Example 6, each cell line was catalogued (yes or no) for the presence of mutations in the following genes by consulting the OncoExpress™ database (Crown Biosciences Inc., Santa Clara, data accession date 18 May 2018˜30 May 2018).
MLH1 (part of the deficient mismatch repair—dMMR-mutations group)
MSH2 (part of the deficient mismatch repair—dMMR-mutations group)
PMS2 (part of the deficient mismatch repair—dMMR-mutations group)
PARP1
BRCA1 (part of the BRCA1/BRCA2 mutations group)
BRCA2 (part of the BRCA1/BRCA2 mutations group)
TP53

Each of these genes encode for some of the important DNA repairing enzymes, and hence the cell lines carrying the mutations in any one or combinations of these genes may be expected with genetic instability.

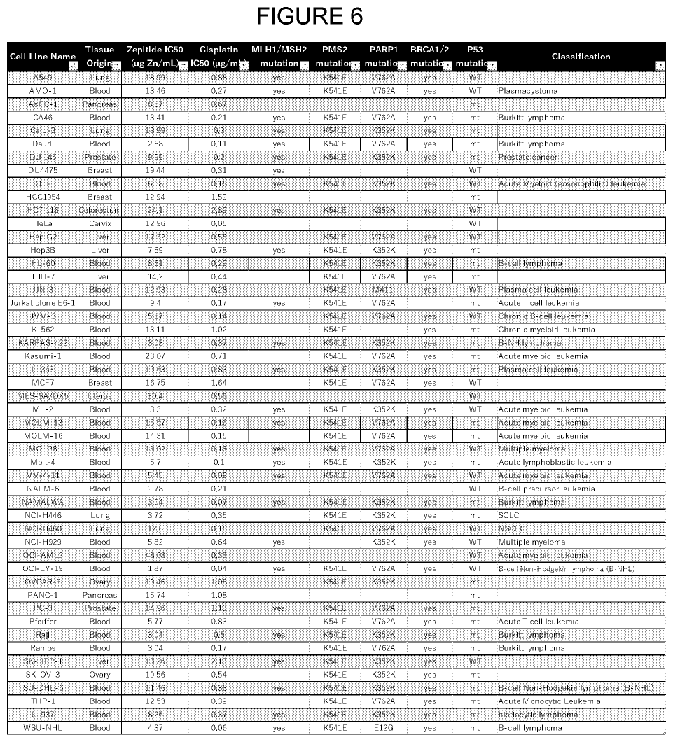

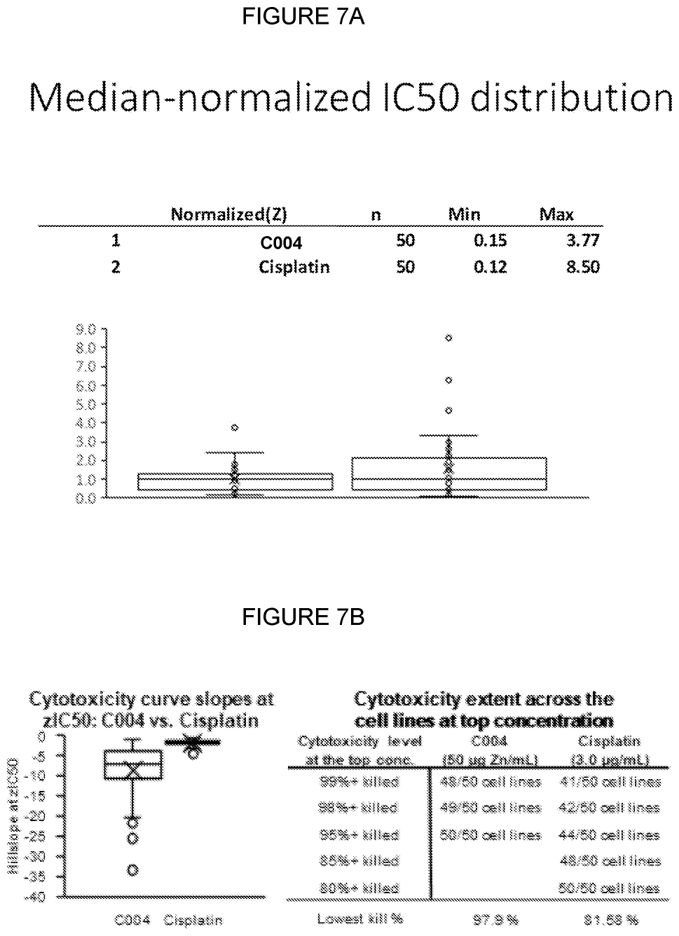

The integrated data table of the Zn/γ-PGA and Cisplatin IC50 values and the cell line gene mutation data across the selected 50 cell lines is shown in FIG. 6. The integrated data were then analyzed using Excel® software (Microsoft, Seattle). Briefly, the sorting algorithm of the Excel® software was used in grouping of the cell lines carrying certain mutations, and in further analysis of the IC50 distribution in each group.

The IC50 distributions among the cell line groups sharing certain genetic mutations were then analyzed for statistical significance.

Statistical analysis was performed using JMP 13 (SAS Institute Inc. Cary, N.C., USA) or Origin 9 (Origin Lab Corp., Northampton, Mass., USA). All data are represented as mean±standard error of the mean (SEM) and were tested for statistical significance using Mann-Whitney U test or analysis of variance (ANOVA). P-values of <0.05 were interpreted as statistically significant.

Results and Analysis.

Stratifying the cell line panel makeup, the 50 cell lines included 30 blood cancers (17 leukemia and 13 lymphoma cell lines) and 20 solid tumor cancers, including breast (3), cervical (1), colorectum (1), liver (4), lung (4), ovarian (2), pancreas (2), prostate (2), and uterine (1) cancer cell lines.

Comparing the results obtained for the Zn(II) agent against the current treatment gold standard for broad spectrum chemotherapy, cisplatin, the Zn(II) agent showed more than 99% eradication in 48 (out of 50) cell lines and more than 97% eradication in all 50 cell lines, versus more than 99% eradication in only 39 (out of 50) cell lines and more than 97% eradication in 45 cell lines for cisplatin. Conversely, cisplatin showed a poor response of less than 90% eradication in 2 cell lines, whereas none of the cell lines exhibited a poor response with the Zn(II) agent.

The 100% response rate and the low Z-value spread (see FIG. 6) for treatment with the Zn(II) agent demonstrates the broad spectrum applicability for Zn(II) agents across all the various cancer types. In particular, the data demonstrate that the Zn(II) agent is more effective than the current approved treatment gold standard against cancer cell types that host mutations in one or more of the following DNA-repair genes: dMMR (MLH1/MSH2/PMS2), PARP1, BRCA1/BRCA2, and TP53. A significant majority of cancer cells carry mutations in one or more of these genes (estimates are of ˜90-95% of all cancers), thus indicating that the methods disclosed herein are a broad spectrum therapeutic for cancer.

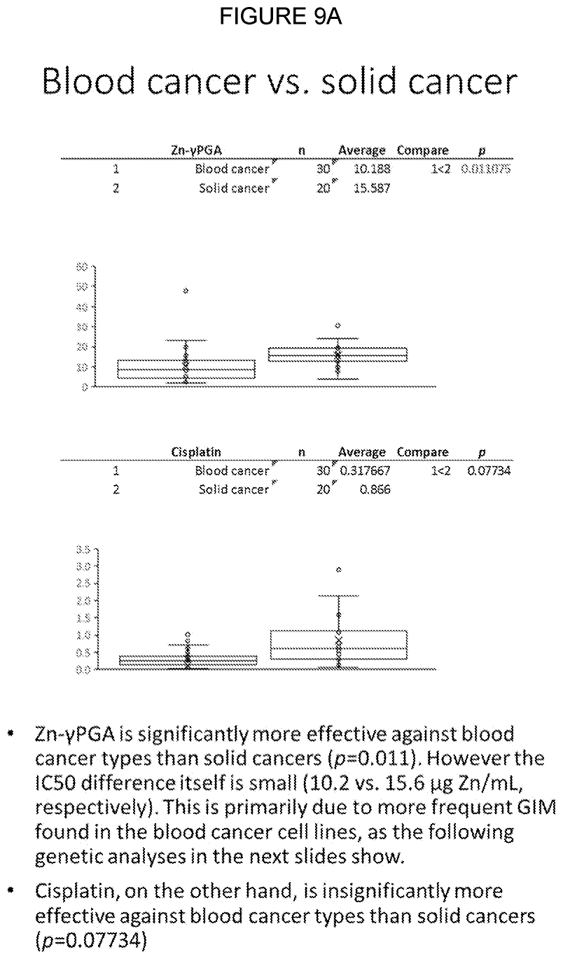

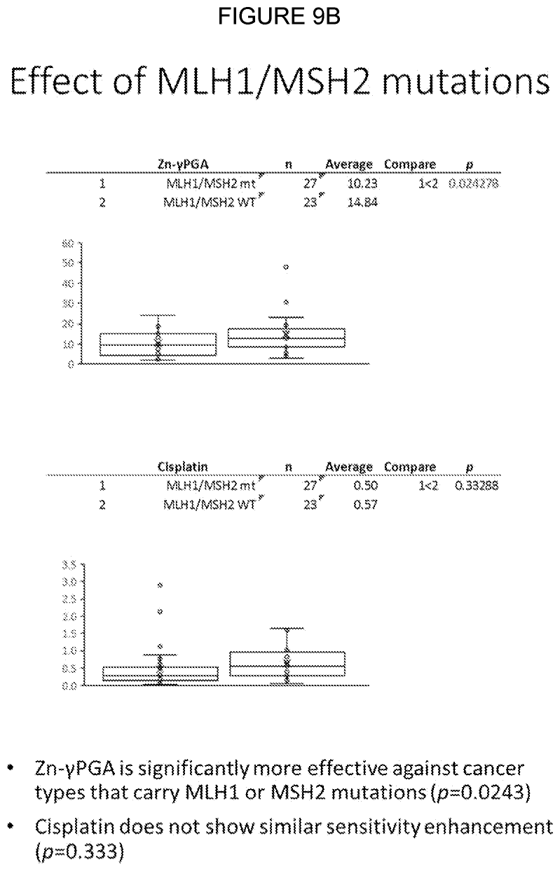

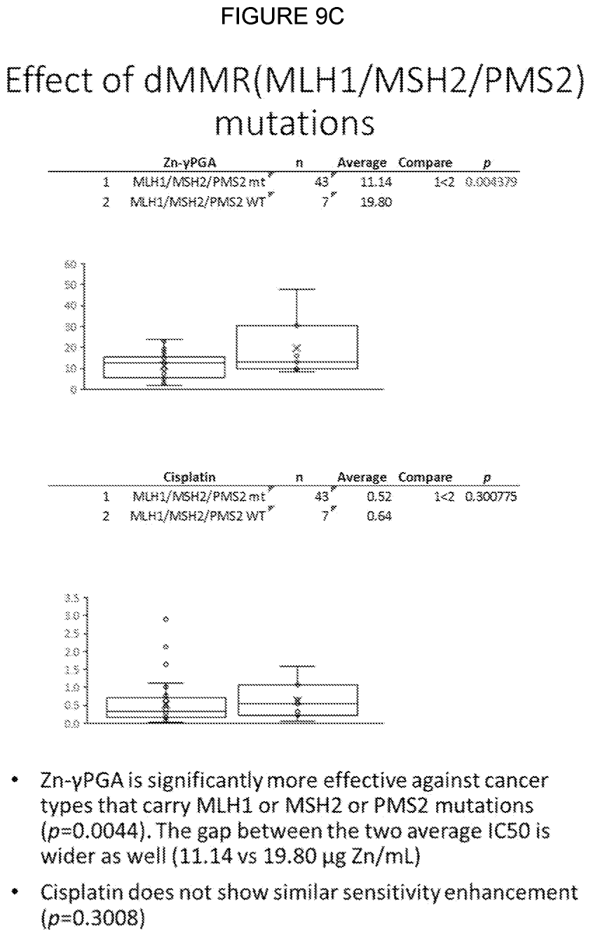

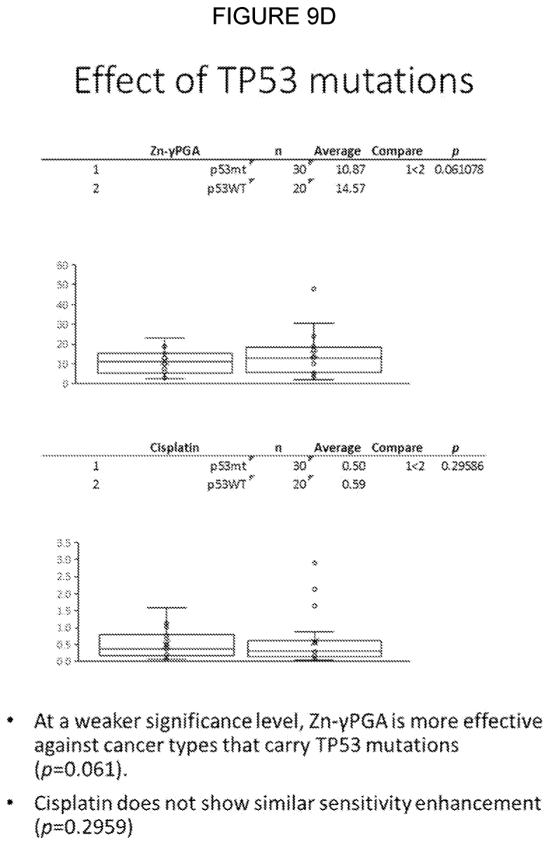

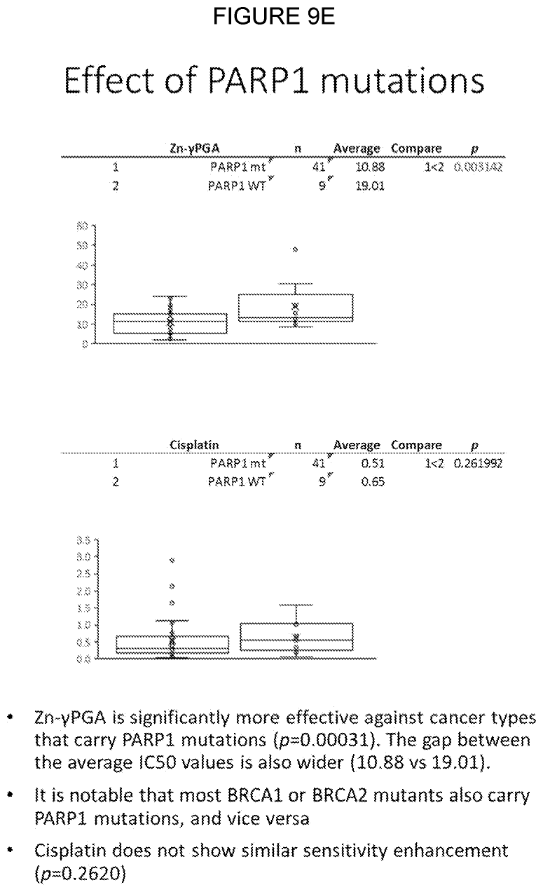

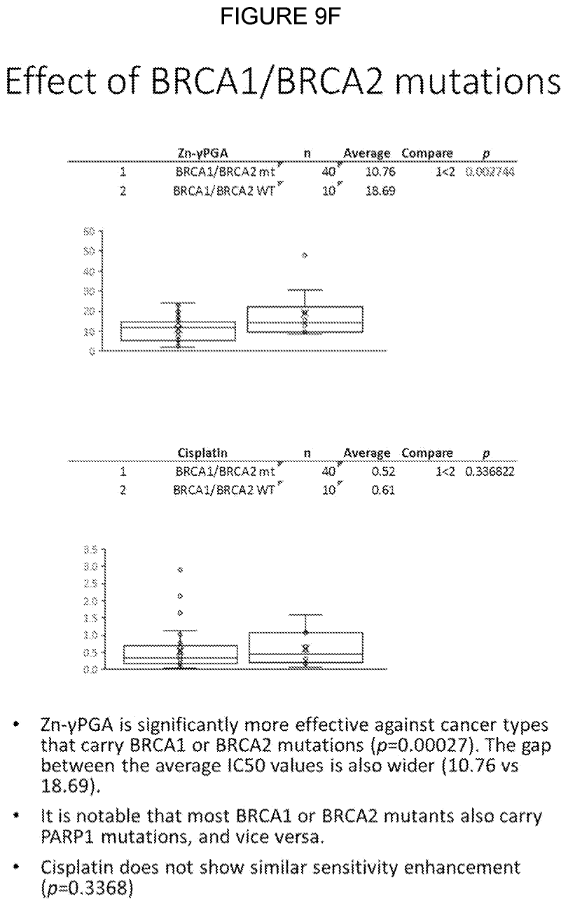

The data analysis for the entire set of cell lines, for the blood cancers vs. solid cancers, and for the cell lines as stratified by mutation, are shown in FIGS. 9A-9F and 10M-10P.

### Example 8: Biomarker Discovery Based on the 50 Cell Line Screen

The expression and mutation were derived from RNAseq data. The RNAseq raw data of the 50 cell lines were downloaded from CCLE database, and SRA database (SRR6799773 for HeLa cell line). The gene copy number results were downloaded from the CCLE website. The driver mutation was predicted on the Cancer Genome Interpreter database (www.cancergenomeinterpreter.org/home), wherein only the driver mutations were used for mutation analysis. Welch's t-test was used to compare the average log₂(IC50) between gene deleted/amplified/mutated and wild type cell lines, and in identifying genes of significance. Spearman correlation test was used to check the correlation between gene expression level and log₂(IC50). The signature genes were selected from genes of significance using Boruta package in R. A linear predictor score (LPS) for each cell line of the form LPS(X)=ΣajXj was calculated, where Xj represents the gene expression of gene j, and aj is the t statistics generated by t-test between sensitive and insensitive cell lines. The mean and variance of the LPS distribution in sensitive and insensitive groups were estimated, and the likelihood that a cell line in which group (sensitive or insensitive) was estimated by applying Bayes' rule so that

$$P ( X in group 1 ) = \frac{∅ ( LPS ( X ) ; {μ}_{1} , {σ}_{1}^{2} )}{∅ ( LPS ( X ) ; {μ}_{1} , {σ}_{1}^{2} ) + ∅ ( LPS ( X ) ; {μ}_{2} , {σ}_{2}^{2} )}$$

Where Ø(x; μ,σ2) represents the normal density function with mean p, and variance σ² and μ1, σ12, vμ₂, σ22 are the observed mean and variance of the LPSs within group 1 and group 2, respectively. All statistical analyses were done with R.

Initial biomarker search using the screening data via RNA sequence-based computerized bioinformatic approaches yielded ADAM6^{del}, CREBBP^{mt}, and PIK3CA^{WT} as the potential biomarkers for C004 sensitivity (FIGS. 12A-12C).

### Example 9: In Vivo Testing of Zn(II) Agents

Zn(II) agent C004: Six-day repeat toxicity test was conducted with daily intravenous injection of C004 at doses up to 2.5 mg Zn/kg/day. While no toxicity in the CT26 tumor bearing BALB/c mice was observed, no significant therapeutic activity (p=0.072) was recorded. (FIG. 13).

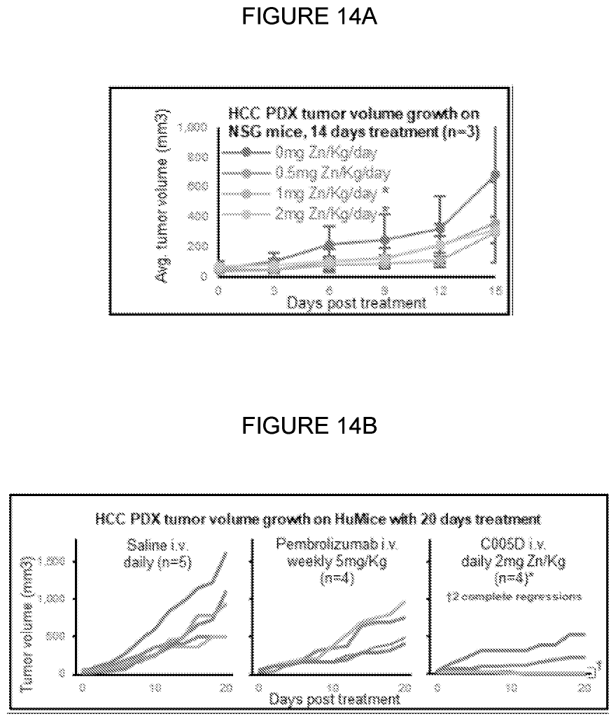

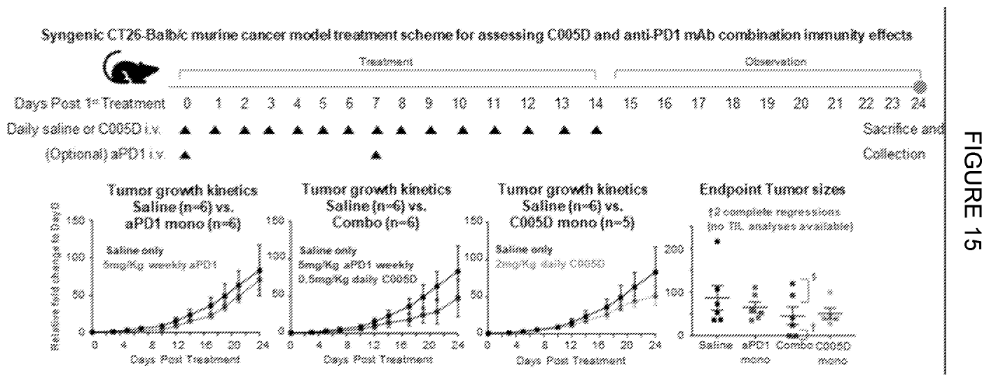

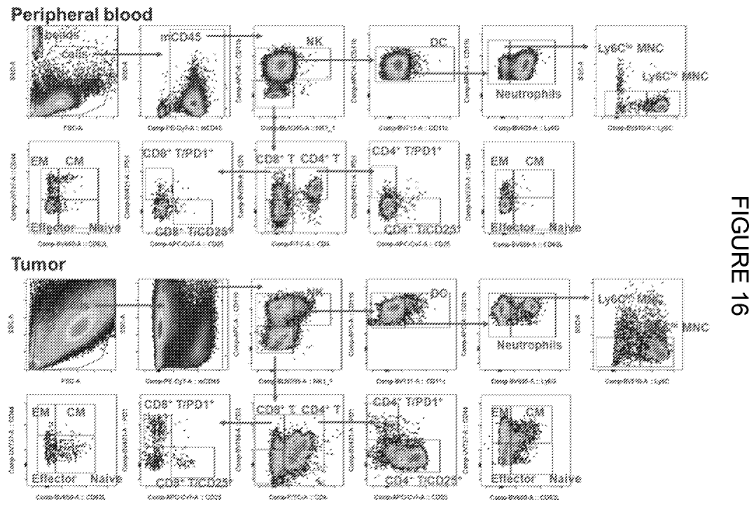

Zn(II) agent C005D: Significant therapeutic activity against human patient-driven hepatocellular carcinoma (HCC PDX-NSG) in immunodeficient in vivo model of NSG mice bearing was observed (FIG. 14A) for daily injection doses of 1 mg Zn/kg/day or 2 mg Zn/kg/day (*p<0.05). In addition, administering C005D against the HCC PDX on humanized immunity mice (HCC-PDX-HuMice) at 2 mg Zn/kg/day resulted in significant tumor suppression effects, with observation of complete tumor regression in two animals at the end of the 20 day treatment period (FIG. 14B).

### Example 10: Monotherapy and Immune-Oncology Agent Combination Therapy and Immunotherapeutic Interactions

Immunocompetent BALB/c mouse bearing CT26 murine cancer were treated with monotherapy arms of a PD-1 inhibitor, aPD1 (5 mg/kg, once weekly i.v.) or C005D (2 mg Zn/kg, daily i.v.) and a combination therarpy arm using a lower dosage of C005D (0.5 mg Zn/kg C005D, daily i.v.+5 mg/kg aPD1, weekly i.v.) in a protocol of 14 days treatment followed by 10 days observation. The protocol summary, the tumor growth kinetics for each arm, and the endpoint tumor sizes are provided in FIG. 15.

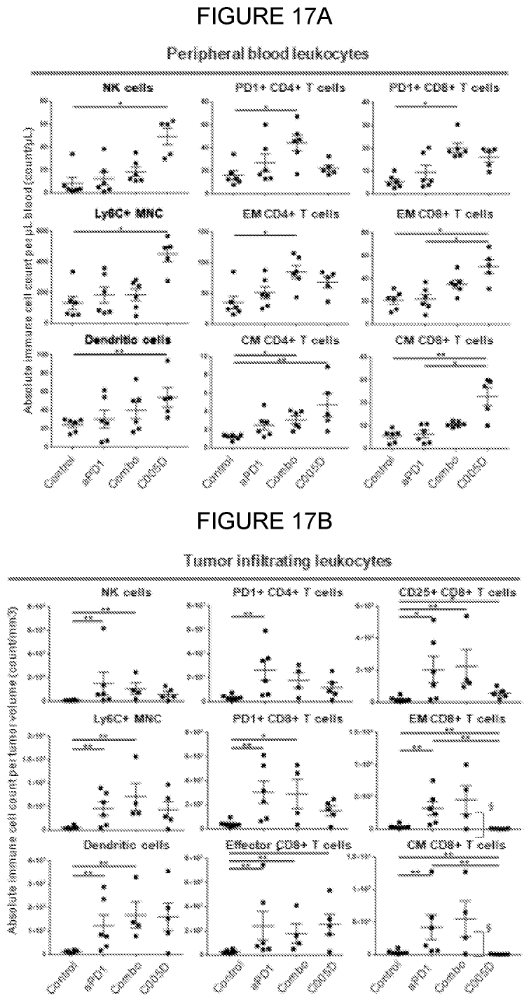

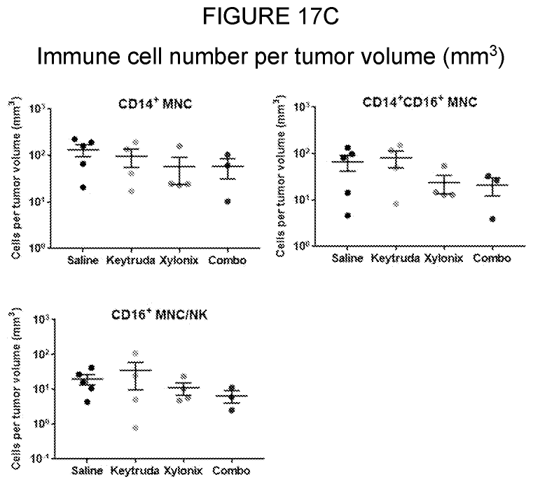

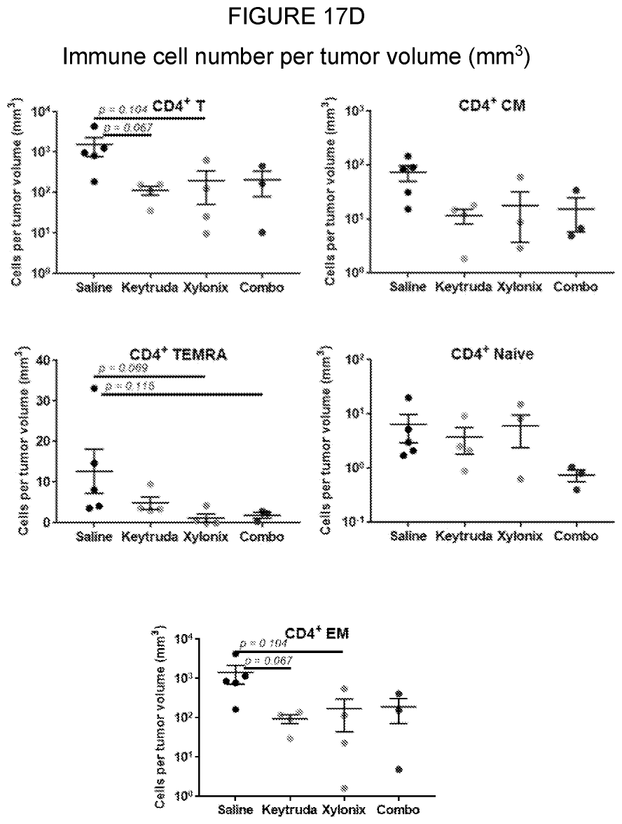

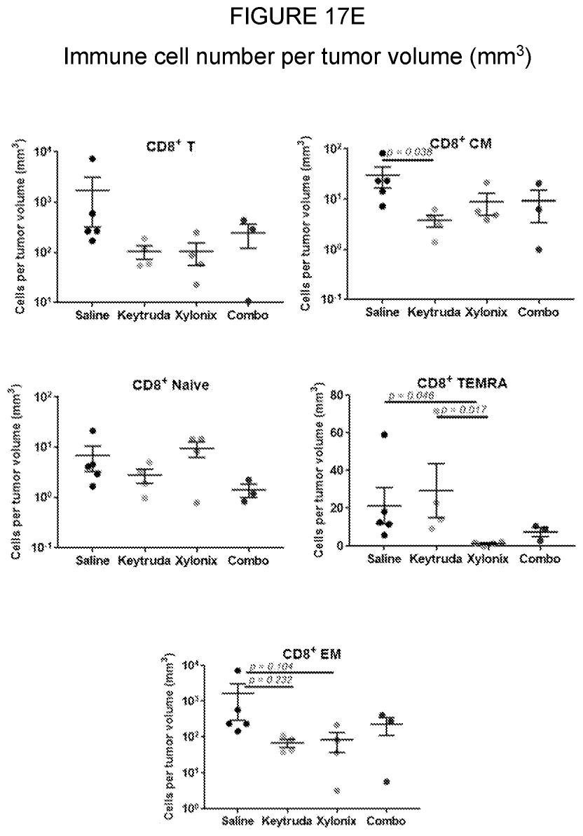

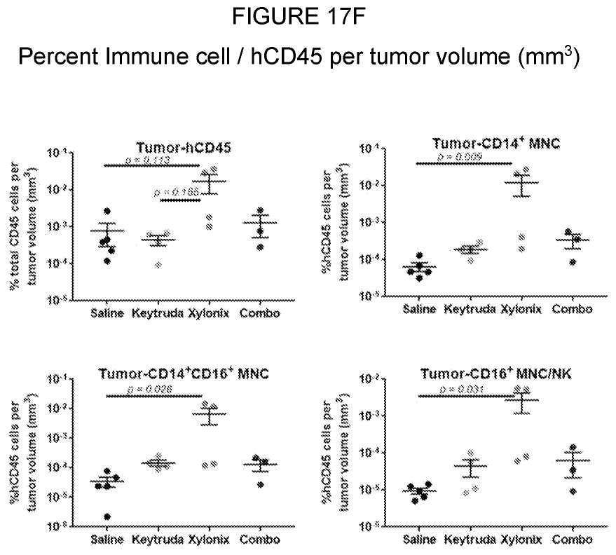

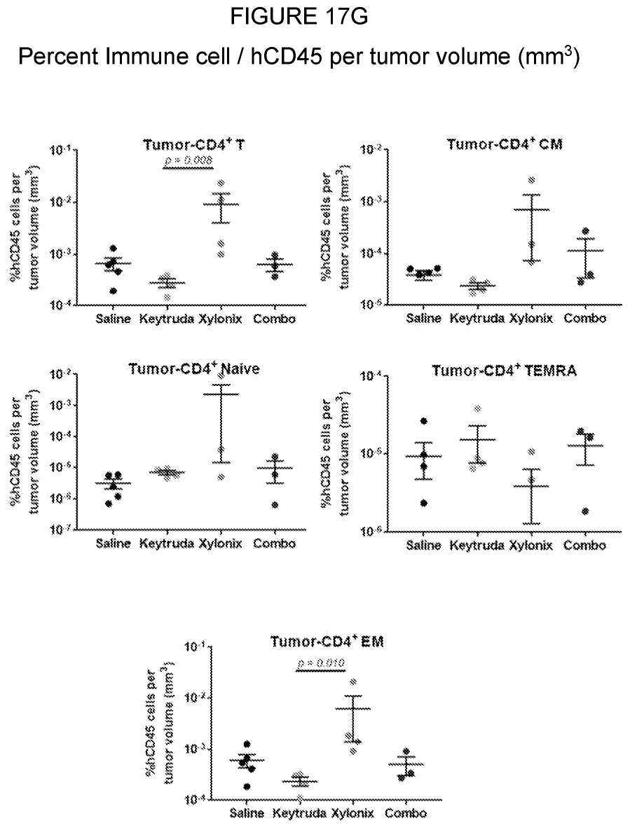

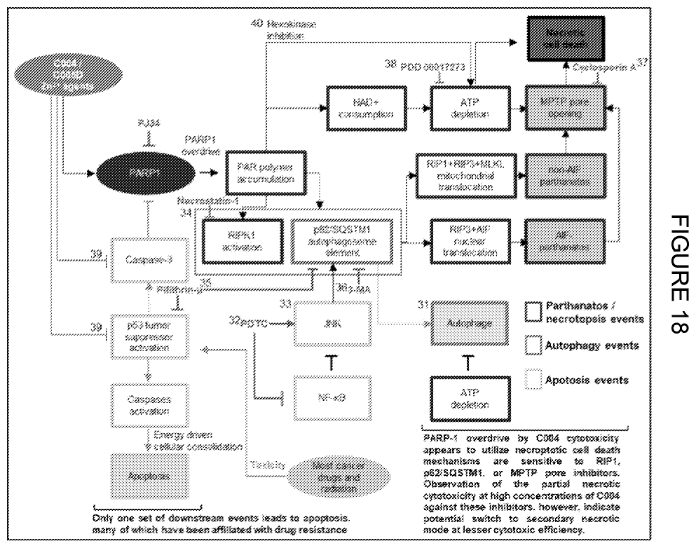

Neither monotherapy arm of aPD1 (5 mg/Kg, once weekly i.v.) or C005D (2 mg Zn/Kg, daily i.v.) produced statistically significant tumor growth suppression effect. However, terminal point immunity characterization from peripheral blood samples and collected tumors revealed distinct differences in the immunity between the two monotherapy arms. The gating strategy for the immunity characterization in shown in FIG. 16. Specifically, significant (*p<0.05) immune-stimulatory effects of aPD1 monotherapy was confined to intratumoral space, whereby NK cells, Ly6C+ monocytes (MN), dendritic cells, and all studied CD4+ T cell subsets and CD8+ T cell subsets were elevated. (See FIG. 17A-17B.) C005D monotherapy, on the other hand, produced significant elevation of immunity in both the peripheral blood compartment, and to a lesser extent, in the intratumoral space.

The combination treatment arm, however, resulted in more pervasive and significant (*p<0.05, **p<0.01) escalation of immunity in both peripheral and intratumoral compartments than either monotherapy arm. Specific to the combination arm, the intratumoral level of the memory cells EM CD8+ T cells and CM CD8+ T cells showed an inverse relationship to the tumor burden of the mice, while two cases of complete tumor regression were also noted in the same group.

The results of the trial indicate the Zn(II) agent combination therapy results in immune-stimulation and synergism with the PD1 blockage. In addition, these results suggest that the combined use of aPD1 and C005D might accelerate formation of the specific CD8+ T cell memory needed for tumor clearance.

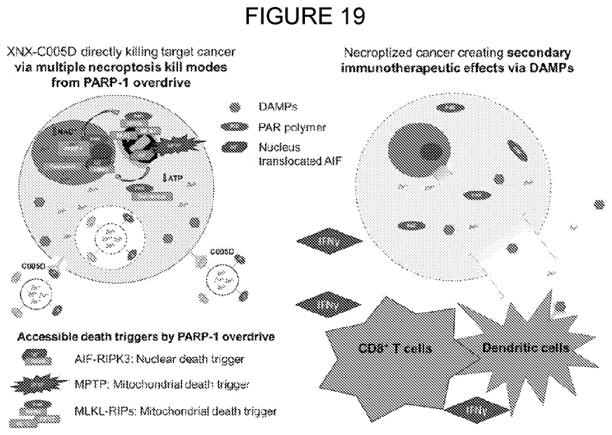

A schematic illustration of the events thought to occur in a tumor cell when Zn(II) agent C005D is administered is shown in FIG. 19. Zinc(II) ions released from a Zn(II) agent elicits PAR polymer accumulation by overdriving PARP-1 while simultaneously conferring PARP-1 protection from caspase-3. In turn, the PAR polymer production and accumulation enable the access to multiple necroptosis kill modes including AIF-mediated nuclear necroptosis, MLKL-mediated mitochondrial necroptosis, and MPTP-mediated mitochondrial necrosis. Continuing to the right side of the figure, the necrosis from PARP-1 overdrive confers secondary immunotherapeutic effect by priming the CD8+ T cell tumoricidal activity via DAMPs release. While the events depicted are consistent with the disclosure herein, the composition, formulations, and treatment methods of the invention are not bound by or limited by the theory espoused in the figure.

### Example 11: Hepatocellular Carcinoma Patient-Derived Xenografts in Humanized Mice Testing and Tumor Infiltrating Leukocyte Analysis

Experimental Drugs.

Pembrolizumab 25 mg/mL (Keytruda®, Merck® KGa) was purchased from Merck®. Isotonic zinc sulfate gamma-polyglutamate solution was prepared at pH 7.0 using Tris buffer (1 mM), at elemental zinc concentration of 1 mg/mL and gamma-polyglutamate concentration of 10 mg/mL.

NSG and Humice.

All manipulations and procedures with mice were approved by Agency for Science, Technology and Research (A*STAR) Institutional Animal Care and Use Committee (IACUC). The diet provided was irradiated TEKLAD GLOBAL 18% Protein Rodent Diet (2918). Mice were housed in a sterile environment and only accessed under a BSL2 hood. Mice were fed, given water and monitored daily for health, and cages were changed weekly. NSG mice were purchased from The Jackson Laboratory and bred in a specific pathogen free facility at the Biological Resource Centre (BRC) in A*STAR, Singapore. One to three days old NSG pups were irradiated with a 55 s exposure equaling 1.1 Gy and transplanted with 1×105 CD34+ human fetal liver cells by intra-hepatic injections. The mice were bled at 8 weeks post-transplantation to determine the fraction of human immune cell reconstitution. Reconstitution was calculated by [% hCD45+/(% hCD45++% mCD45+)]. 8-10 weeks old humanized mice reconstituted with 20-50% of human CD45+ cells were used for engraftment.

HCC-PDX Tumor Maintenance and Xenografts.

For in vivo HCC-PDX subcutaneous humanized model establishment, patient HCC tumors were collected from HCC surgical specimens. Before surgery, all patients gave written informed consent for their HCC samples to be used for research. After appropriate clinical tissue is taken, the remainder of the HCC is transferred on ice with media consisting of DMEM containing 10% FCS, 1% penicillin/streptavidin to where the PDX is to be established. Within 4 hours, HCC fragments were cut into pieces of ca. 3×3×3 mm using sterile surgical instruments. Once the mice are anaesthetized, and shaved, for subcutaneous placement, using forceps to lift up the skin to ensure no peritoneal violation a small 1 cm incision is made in the skin with scissors. The subcutaneous is probed to create a pocket, the tissue is placed inside the pocket and the skin is closed with adhesive, suture or clips.

To maintain HCC-PDX tumors in NSG mice, HCC obtained from the first generation of mice (P1) were serially transplanted to the next cohorts of mice (P2 and P3). HCCs were harvested from established PDXs were cut into pieces of ca. 3×3×3 mm3 using sterile surgical instruments in a laminar flow cabinet. Pieces were transferred into sterile cryotubes containing 1.5 mL 95% FCS/5% DMSO. Cryotubes were put in CoolCell container (Biocision), placed in a −80° C. freezer overnight and transferred to liquid nitrogen storage the next day. For thawing, cryotubes were held in a water bath (37° C.) until melted.

Determination of Tumor Size.

Tumor volume was measured in two dimensions (length and width) using calipers and the tumor volume was calculated using the formula: Tumor size=(length 2×width)×½.

Isolation of leukocytes from blood, spleen, bone marrow and HCC-PDX tumor.

150-200 μl blood was collected in potassium EDTA MiniCollect® tubes (Greiner bio-one, 450475) via cheek bleeding. 30 μl of blood mixed with 20p1 CountBright™ Absolute Counting Beads (ThermoFisher) were plated in 96-well V-bottom plates at room temperature before processing and data acquisition. Fresh spleen was excised from mouse and placed in PBS on ice. Crush spleen through a 100 μm cell strainer (Falcon) using a syringe plunger until only connective tissue is left. For bone marrow cells isolation, tibias, femurs, hip and spine were dissected from mice. The clean bones are crushed with mortar and pestle in medium (PBS+2% FCS+2 mM EDTA). The cell mixture obtained from each mouse is kept separate and filtered through a 100 μm cell strainer (Falcon). All samples were processed within one hour of collection. To isolate the TILs from HCC, tumor was cut up into 1-2 mm2 fragments after trimming away fat and connective tissue and disaggregated with human tumor dissociation Kit (Miltenyi Biotec) using gentleMACS™ Dissociator (Miltenyi Biotec). The cell suspension was filtered through a 100 μm cell strainer (Falcon). Cell suspension layered over a discontinuous 40% followed by a 80% Percoll® Density Gradient Media (GE Healthcare). Leukocytes are located at the interface between 40% and 80% Percoll. The enriched TILs were then washed in D-PBS, 1% BSA and then processed as the described.

Flow Cytometry.

Cell mixture from blood, spleen, bone marrow and tumor was suspended in ammonium chloride-potassium (ACK) lysing buffer (Life Technologies) and incubated for 10 minutes at room temperature with gentle mixing to lyse contaminating red blood cells (RBC). For surface staining, leukocytes were washed twice in Fluorescence-activated cell sorting (FACS) buffer [Phosphate-buffered saline (PBS)+2% BSA+1 mM EDTA+0.1% sodium azide], incubated with Fc blocking reagent (Miltenyi Biotec) and stained with directly conjugated antibodies. For intracellular staining, blood leukocytes were labeled with surface markers as previous described and then fixed and permed with Transcription Factor Buffer Set (BD Pharmingen™) Five antibodies panel were used for this study. Human T cell panel (15 colors): hCD4-BUV395, hCD8-BUV373, hCD183-BV421, hCD197-BV510, hCD25-BV605, hCD196-BV650, hCD38-BV711, hCD45RO-BV785, hCD45RA-FITC, hCD127-PE, hCD194-PE-CF594, hCD3-PERCP5.5, hCD185-PE-CY7, hCCR10-APC and hHLA-DRAPC-CY7. Human Non T cell panel (15 colors): hCD45-BUV395, hCD19-BUV373, hCD56-BV421, hIgD-BV510, hCD11c-BV605, hCD27-BV650, hCD38-BV711, hCD16BV785, hCD123-FITC, hCD20-PE, hCD24-PE-CF594, hCD66b-PERCP5.5, hCD3-PE-CY7, hCD14-APC and hHLA-DR-APC-CY7. Human Tex cell panel (15 colors): hCD4-BUV395, hCD8-BUV373, hCD272-BV421, hCD197-BV510, hKLRG-1-BV605, hCD28-BV650, hCD279-BV711, hCD366-BV785, hCD45RA-FITC, hCD57-PE, hCD152-PE-CF594, hCD160-PERCP5.5, hTIGIT-PE-CY7, hCD223-APC and hCD244-APC-CY7. Human Tc cell panel (11 color): hCD4-BUV395, hCD8-BUV373, Granzyme B-BV421, hCD197BV510, hIFN-γ-BV605, hTNF-α-BV650, hCD3-BV785, hCD45RA-FITC, Granulysin-PE, Granzyme A-PERCP5.5, Perforin-APC and hIL-2-APC-CY7.TAM and MDSC panel (13 color): hCD45-BUV395, hCD11b-BV421, hCD86-BV605, hCD15-BV650, hCD204-BV711, hCD16-BV785, hCD33-FITC, Lineage (hCD3, hCD19 and hCD56)-PE, hCD68-PE-CF594, hCD163-PERCP5.5, hCD124-PE-CY7, hCD14-APC and hHLA-DR-APC-CY7. Dead cell exclusion was performed with the addition of DAPI (Life Technologies).

Human Multiplex Cytokine Analysis.

Plasma cytokines were analyzed using the LEGENDplex™ human Th Cytokine Panel (13plex) array kit, human cytokine Panel 2 (13-plex) array kit and human CD8/NK panel assay kit (13-plex) from Biolegend according to the manufacturer's protocol. The data were collected on a LSR II flow cytometer and analyzed using the LEGENDplex™ software version 7.0 (Biolegend).

Control and Experimental Drug Treatments.

Once the HCC-PDX xenograft was established at approximate tumor volume of 100 mm³ on the Humanized NSG mice, control and experimental drugs treatments were started. Tumor volumes were measured on every 2 days until the termination of the study (21 days post-first treatment [dpf]). The treatment schedules were as the following.
Saline treatment (Control group): Daily 100 μL saline injection via tail vein.
Pembrolizumab only (Keytruda group): A bolus injection at 5 mg/kg via tail vein at 0 dpf ONLY.
XYLONIX Zn-γPGA treatment (Xylonix group): Daily injection via tail vein at the dose of 2 mg Zn/Kg/day.
Pembrolizumab+Xylonix (Combo group): the combination of the above two treatment regimens.

Statistical Analysis.

Statistical analysis was performed using JMP 13 (SAS Institute Inc. Cary, N.C., USA) or Origin 9 (Origin Lab Corp., Northampton, Mass., USA). All data are represented as mean±standard error of the mean (SEM) and were tested for statistical significance using Mann-Whitney U test or analysis of variance (ANOVA). P-values of <0.05 were interpreted as statistically significant.

Results and Analysis

The growth kinetics study clearly showed superior antitumor efficacy of Zn-γPGA mono (daily iv) over saline, pembrolizumab mono (bolus at the start), or its combination with pembrolizumab. TIL analysis findings supported the growth kinetics by showing that Zn-γPGA monotherapy stimulated both CD4+ T cells and CD8+ T cells, while pembrolizumab mono or combo efficacy were solely dependent on CD8+ T cells.

The total tumor infiltrating leukocyte (TIL) analyses are shown in FIGS. 17C-17G. The immune cell activity index, which was calculated using the following formula, is shown for various immune cell types in FIGS. 17F and 17G.

$$Total tumor infiltrating leukocyte analyses \text{:}$$

$$Immune cell activity index = \frac{\# Target human immune cell}{\# hCD 45 + cells} \times \frac{1}{Tumor Volume ( mm 3 )}$$

Patient-derived xenografts are known to faithfully conserve the genetic patterns of the primary tumors, and studies have shown that screening studies of the type demonstrated herein correlate with patient outcomes, and thus the model demonstrates treatments that have clinical benefit.

The following examples illustrates Zn(II) agents prepared from Zn(II) salts and either gamma-polyglutamic acid (γ-PGA) or alpha-glutamic acid (α-PGA).

### Example 12: Preparation and Characterization of Zn/γ-PGA at pH 7.0 Using Phosphate-Precipitation Method for Removing Non-Bound Excess Zinc

To prepare Zn/γ-PGA, 55 mg PGA (50,000 Da molecular weight) was dissolved in 5 mL 10 mM MES buffer, pH 7.0, containing 10 mM ZnSO₄ at room temperature, and then sonicated while placed on ice for 10 minutes. Then, 0.5 mL 200 mM phosphate buffer, pH 7.0, was added to the solution to precipitate free zinc ions, and the mixture was filtered through a 0.2 μm syringe sterilization filter. The zinc content was measured using ICP-MS and by 4-(2-pyridylazo)-resorcinol assay. The final stock Zn/γ-PGA contained 1% (wt/vol) PGA and 400 μg/mL bound zinc ions. Stock Zn/γ-PGA solutions were prepared fresh on each day of administration.

### Example 13: Preparation and Characterization of Zn/γ-PGA at pH 7.0 Using Dialysis Method for Removing Non-Bound Excess Zinc

To prepare ZnPGA, 55 mg PGA (50,000 Da molecular weight) was dissolved in 5 mL 10 mM MES buffer, pH 7.0, containing 10 mM ZnSO₄ at room temperature, and then sonicated while placed on ice for 10 minutes. Then, the solution was dialyzed on ice against 1 L 10 mM MES, pH 7.0, for 2 hours, successively three times, for a total of 3 volumes over 6 hours. The recovered solution was filtered through a 0.2 μm syringe sterilization filter. The zinc content was measured using ICP-MS and by 4-(2-pyridylazo)-resorcinol assay. The final stock Zn/γ-PGA contained 0.9% (wt/vol) PGA and 380 μg/mL bound zinc ions. Stock Zn/γ-PGA solutions were prepared fresh on each day of administration.

### Example 14: Liquid Formulation

The composition of an exemplary embodiment of liquid formulation suitable for, e.g., injection comprises a zinc(II) salt, γ-PGA, sodium chloride, and water. The composition is prepared by combining zinc sulfate heptahydrate, γ-PGA (potassium salt, 100 kDa), sodium chloride and adding water to volume, wherein the concentrations of each component are 1 mg/mL zinc(II), 10 mg/mL γ-PGA, and 6.5 mg/mL sodium chloride. The resulting composition of approximately 276 mOsm/kg osmolality and pH 5.68 is suitable for injection in human patients.

### Example 15: γ-Polyglutamic Acid-Zinc Liquid Composition

A composition useful for performing the invention according to an embodiment is shown in Table 3. The composition provides 0.68 mg of Zn (Zn²⁺ ion) per 100 g as a liquid suspension formulation comprising wax-coated particles. A method for preparing the formulation follows the table. This composition is merely illustrative of one of many compositions useful for the subject invention.

TABLE 3

| Suspended Solid Components | Amount | |
|---|---|---|
| Zinc sulfate•7H2O | 3.011 | mg |
| γ-PGA (MW(Mₙ) ≤ 100 kDa) | 6.848 | mg |
| Sucrose | 9.5107 | g |
| HPMC-P | 0.3804 | g |
| Wax | 98.91 | mg |
| SUBTOTAL | 10 | g |
| Solution Components | Amount | |
| Xanthan gum | 0.3 | g |
| Guar gum | 0.3 | g |
| Xylitol | 10 | g |
| Citric acid | 0.5 | g |
| Limonene | 0.1 | g |
| Potassium sorbate | 0.025 | g |
| Water | 78.7 | mL |
| TOTAL | 99.925 | g |

A. Preparation of coated Zn/γ-PGA microspheres (cZPM). 200 mL water containing 10 g sucrose (5% w/v), 45 mg γ-PGA, and 19.79 mg zinc sulfate heptahydrate (4.5 mg as elemental Zn) was prepared and freeze-dried. The resulting powder was then triturated in a 1:4 ratio with finely divided sucrose containing up to 5% cornstarch and pressed through a No. 50 U.S. Standard stainless steel sieve (48 Mesh). This powder was then suspended in 200 mL of white paraffin oil in a 400 mL beaker. The mixture was dispersed by stirring at 260 rpm with a 44 mm polyethylene three-blade paddle fitted to a high-torque stirrer (Type RXR1, Caframo, Wiarton, Ontario). To the suspension was added 20 mL of 10% (w/v) hydroxypropylmethylcellulose-phthalate (HPMC-P) in acetone-95% ethanol (9:1). Stirring was continued for 5 min, whereby microspheres form, and then 75 mL of chloroform was added. The suspending medium was decanted, and the microspheres were briefly resuspended in 75 mL of chloroform, and air-dried at ambient temperature. Upon drying, the microspheres were coated with Carnauba wax. Specifically, 1 g of Carnauba wax was dissolved in 200 mL of white paraffin oil at 70° C., and cooled to less than 45° C. To this cooled wax-paraffin solution, the prepared microspheres were added and suspended for 15 minutes with constant stirring. The wax solution was then decanted, and the microspheres were collected on filter paper to absorb the excess wax solution to obtain coated Zn/γ-PGA microspheres (cZPM).

B. Preparation of liquid suspension solution of coated Zn/γ-PGA microspheres (cZPM). The following components: 0.3 g xanthan gum (e.g., as a suspending polymer); 0.3 g guar gum (e.g., as a viscosity agent); 10 g xylitol (e.g., as a sweetener); 0.5 g citric buffer (e.g., as a buffer); 0.1 g limonene (e.g., as a flavoring agent); 0.025 g potassium sorbate (e.g., as a preservative), were dissolved in 78.7 mL water. The pH of the aqueous solution was adjusted to pH 4.5, and then 10 g cZPM was suspended in the aqueous solution to obtain the cZPM liquid suspension.

### Example 16: γ-Polyglutamic Acid-Zinc Composition

A composition useful for performing the invention according to an embodiment is shown in Table 4. The composition provides 25 mg of Zn (Zn²⁺ ion) per tablet. A method for preparing the tablet follows the table. This composition is merely illustrative of one of many compositions useful for the subject invention.

TABLE 4

| | Amount per | |
|---|---|---|
| Component | tablet | Weight % |
| Zinc sulfate | 110 mg | 22% |
| γ-Polyglutamic acid | 110 mg | 22% |
| Microcrystalline cellulose | 100 mg | 20% |
| Starch | 85 mg | 17% |
| Silicon dioxide | 50 mg | 10% |
| Magnesium stearate | 25 mg | 5% |
| Cellulose acetate phthalate | 20 mg | 4% |
| Total | 500 mg | 100% |

Coated tablets with the composition shown in Table 2 may be prepared using a wet granulation technique. First, zinc sulfate and γ-polyglutamic acid are mixed together dry. Microcrystalline cellulose, starch, and silicon dioxide are further added, and the dry components are all further mixed together. The mixed components are transferred to a granulator and an appropriate amount of aqueous ethanol is added and granulation is carried out. The obtained granulated mixture is dried at 50-70° C. to yield a granulated composition with less than about 5% water content. Magnesium stearate is added to and mixed with the granulated composition. The obtained mixture is compressed into tablets. Finally, the tablets are coated with cellulose acetate phthalate using standard techniques, as known to those skilled in the art.

### Example 17: γ-Polyglutamic Acid-Zinc Composition

A composition useful for performing the invention according to an embodiment is shown in Table 5. The composition provides 30 mg of Zn (Zn²⁺ ion) per tablet. A method for preparing the tablet follows the table. This composition is merely illustrative of one of many compositions useful for the subject invention.

TABLE 5

| | Amount | | |
|---|---|---|---|
| Component-Tablet core | per tablet | | Weight % |
| Zinc sulfate•7H2O | 132.3 | mg | 26.5% |
| γ-PGA (MW(Mₙ) ≤ 100 kDa) | 132.3 | mg | 26.5% |
| Microcrystalline cellulose | 102.5 | mg | 20.5% |
| HPMC-P | 65.0 | mg | 13% |
| Maltodextrin | 37.9 | mg | 7.6% |
| Carboxymethylcellulose-Ca | 5.0 | mg | 1.0% |
| Aerosil ® | 5.0 | mg | 1.0% |
| Magnesium stearate | 5.0 | mg | 1.0% |
| 70% Ethanol | q.s | | NA* |
| Purified water | q.s | | NA* |
| SUBTOTAL | 485 | mg | |
| Component-Tablet coating | Amount | | Weight % |
| HPMC-P | 10.0 | mg | 2.0% |
| HPMC | 5.0 | mg | 1.0% |
| Isopropyl alcohol | 0.16 | mL | NA* |
| Purified water | 0.13 | mL | NA* |
| TOTAL | 500 | mg | 100% |

*It is assumed here that the solvents (ethanol, isopropyl alcohol, and water) are present in insignificant amounts in the formulated tablet.

Coated tablets with the composition shown in Table 3 may be prepared as follows. First, zinc sulfate, γ-polyglutamic acid, microcrystalline cellulose, HPMC-P (hydroxypropylmethylcellulose phthalate), maltodextrin, and carboxymethylcellulose-calcium were mixed together dry. The mixed components were transferred to a granulator and an appropriate amount of 70% aqueous ethanol was added and wet granulation was carried out. The obtained granulated mixture was dried at up to about 60° C. to yield a granulated composition with less than about 3% LOD (loss on drying). Silica (e.g., Aerosil®) and magnesium stearate was added to and mixed with the granulated composition. The obtained mixture was compressed into tablets. The tablets were first coated using an isopropyl alcohol solution of HPMC-P, and then coated in a second step using an aqueous solution of HPMC, using standard techniques, as known to those skilled in the art.

### Example 18: Preparing and Characterizing Zn/α-PGA at pH 7.0 Using Phosphate-Precipitation Method for Removing Non-Bound Excess Zinc

To prepare Zn/α-PGA, 55 mg α-PGA, sodium salt, 60 kDa average molecular weight (monodisperse) (Alamanda Polymers, Huntsville, Ala.), is dissolved in 5 mL 10 mM MES buffer, pH 7.0, containing 10 mM ZnSO₄ at room temperature, and then sonicated while placed on ice for 10 minutes. Then, 0.5 mL 200 mM phosphate buffer, pH 7.0, is added to the solution to precipitate free zinc ions, and the mixture is filtered through a 0.2 μm syringe sterilization filter. The zinc content is measured using ICP-MS and by 4-(2-pyridylazo)-resorcinol assay. Stock solutions of Zn/α-PGA containing, for example, 1% (wt/vol) PGA and 400 μg/mL bound zinc ions may be prepared and used for oral administration.

### Example 19: Preparing and Characterizing Zn/α-PGA at pH 7.0 Using Dialysis Method for Removing Non-Bound Excess Zinc

To prepare Zn/α-PGA, 55 mg α-PGA, sodium salt, 60 kDa average molecular weight (monodisperse) (Alamanda Polymers, Huntsville, Ala.), is dissolved in 5 mL 10 mM MES buffer, pH 7.0, containing 10 mM ZnSO₄ at room temperature, and then sonicated while placed on ice for 10 minutes. Then, the solution is dialyzed on ice against 1 L 10 mM MES, pH 7.0, for 2 hours, successively three times, for a total of 3 volumes over 6 hours. The recovered solution is filtered through a 0.2 μm syringe sterilization filter. The zinc content is measured using ICP-MS and by 4-(2-pyridylazo)-resorcinol assay. Stock solutions of Zn/α-PGA containing, for example, 1% (wt/vol) PGA and 400 μg/mL bound zinc ions may be prepared and used for oral administration.

### Example 20: Liquid Formulation

The composition of an exemplary embodiment of liquid formulation suitable for, e.g., injection comprises a zinc(II) salt, α-PGA, sodium chloride, and water. The composition is prepared by combining zinc sulfate heptahydrate, α-PGA sodium salt, 60 kDa average molecular weight (monodisperse) (Alamanda Polymers, Huntsville, Ala.), sodium chloride and adding water to volume, wherein the concentrations of each component are 1 mg/mL zinc(II), 10 mg/mL α-PGA, and 6.5 mg/mL sodium chloride. The resulting composition of approximately 276 mOsm/kg osmolality and pH 5.68 is suitable for injection in human patients.

### Example 21: α-Polyglutamic Acid-Zinc Liquid Composition

A composition useful for performing the invention according to an embodiment is shown in Table 6. The composition provides 0.68 mg of Zn (Zn²⁺ ion) per 100 g as a liquid suspension formulation comprising wax-coated particles. A method for preparing the formulation follows the table. This composition is merely illustrative of one of many compositions useful for the subject invention.

TABLE 6

| Suspended Solid Components | Amount | |
|---|---|---|
| Zinc sulfate•7H2O | 3.011 | mg |
| α-PGA (MW(Mₙ) ≤ 100 kDA) | 6.848 | mg |
| Sucrose | 9.5107 | g |
| HPMC-P | 0.3804 | g |
| Wax | 98.91 | mg |
| SUBTOTAL | 10 | g |
| Solution Components | Amount | |
| Xanthan gum | 0.3 | g |
| Guar gum | 0.3 | g |
| Xylitol | 10 | g |
| Citric acid | 0.5 | g |
| Limonene | 0.1 | g |
| Potassium sorbate | 0.025 | g |
| Water | 78.7 | mL |
| TOTAL | 99.925 | g |

A. Preparing coated Zn/α-PGA microspheres (cZPM). 200 mL water containing 10 g sucrose (5% w/v), 45 mg α-PGA, and 19.79 mg zinc sulfate heptahydrate (4.5 mg as elemental Zn) is prepared and freeze-dried. The resulting powder is triturated in a 1:4 ratio with finely divided sucrose containing up to 5% cornstarch and pressed through a No. 50 U.S. Standard stainless steel sieve (48 Mesh). This powder is suspended in 200 mL of white paraffin oil in a 400 mL beaker. The mixture is dispersed by stirring at 260 rpm with a 44 mm polyethylene three-blade paddle fitted to a high-torque stirrer (Type RXR1, Caframo, Wiarton, Ontario). To the suspension is added 20 mL of 10% (w/v) hydroxypropylmethylcellulose-phthalate (HPMC-P) in acetone-95% ethanol (9:1). Stirring is continued for 5 minutes, whereby microspheres form, and then 75 mL of chloroform is added. The suspending medium is decanted, and the microspheres are briefly resuspended in 75 mL of chloroform, and air-dried at ambient temperature. Upon drying, the microspheres are coated with Carnauba wax. Specifically, 1 g of Carnauba wax is dissolved in 200 mL of white paraffin oil at 70° C., and cooled to less than 45° C. To this cooled wax-paraffin solution, the prepared microspheres are added and suspended for 15 minutes with constant stirring. The wax solution is decanted, and the microspheres are collected on filter paper to absorb the excess wax solution to obtain coated Zn/α-PGA microspheres (cZPM).

B. Preparing liquid suspension solution of coated Zn/α-PGA microspheres (cZPM). The following components: 0.3 g xanthan gum (e.g., as a suspending polymer); 0.3 g guar gum (e.g., as a viscosity agent); 10 g xylitol (e.g., as a sweetener); 0.5 g citric buffer (e.g., as a buffer); 0.1 g limonene (e.g., as a flavoring agent); 0.025 g potassium sorbate (e.g., as a preservative), are dissolved in 78.7 mL water. The pH of the aqueous solution is adjusted to pH 4.5, and 10 g cZPM is suspended in the aqueous solution to obtain the cZPM liquid suspension.

### Example 22: α-Polyglutamic Acid-Zinc Composition

A composition useful for performing the invention according to an embodiment is shown in Table 7. The composition provides 25 mg of Zn (Zn²⁺ ion) per tablet. A method for preparing the tablet follows the table. This composition is merely illustrative of one of many compositions useful for the subject invention.

TABLE 7

| | Amount per | |
|---|---|---|
| Component | tablet | Weight % |
| Zinc sulfate | 110 mg | 22% |
| α-Polyglutamic acid (MW(Mₙ) ≤ | 110 mg | 22% |
| 100 kDa) | | |
| Microcrystalline cellulose | 100 mg | 20% |
| Starch | 85 mg | 17% |
| Silicon dioxide | 50 mg | 10% |
| Magnesium stearate | 25 mg | 5% |
| Cellulose acetate phthalate | 20 mg | 4% |
| Total | 500 mg | 100% |

Coated tablets with the composition shown in Table 2 may be prepared using a wet granulation technique. First, zinc sulfate and α-polyglutamic acid are mixed together dry. Microcrystalline cellulose, starch, and silicon dioxide are further added, and the dry components are all further mixed together. The mixed components are transferred to a granulator and an appropriate amount of aqueous ethanol is added and granulation is carried out. The obtained granulated mixture is dried at 50-70° C. to yield a granulated composition with less than about 5% water content. Magnesium stearate is added to and mixed with the granulated composition. The obtained mixture is compressed into tablets. Finally, the tablets are coated with cellulose acetate phthalate using standard techniques, as known to those skilled in the art.

### Example 23: α-Polyglutamic Acid-Zinc Composition

A composition useful for performing the invention according to an embodiment is shown in Table 8. The composition provides 30 mg of Zn (Zn²⁺ ion) per tablet. A method for preparing the tablet follows the table. This composition is merely illustrative of one of many compositions useful for the subject invention.

TABLE 8

| | Amount | | |
|---|---|---|---|
| Component-Tablet core | per tablet | | Weight % |
| Zinc sulfate•7H2O | 132.3 | mg | 26.5% |
| α-PGA (MW(Mₙ) ≤ 100 kDa) | 132.3 | mg | 26.5% |
| Microcrystalline cellulose | 102.5 | mg | 20.5% |
| HPMC-P | 65.0 | mg | 13% |
| Maltodextrin | 37.9 | mg | 7.6% |
| Carboxymethylcellulose-Ca | 5.0 | mg | 1.0% |
| Aerosil ® | 5.0 | mg | 1.0% |
| Magnesium stearate | 5.0 | mg | 1.0% |
| 70% Ethanol | q.s | | NA* |
| Purified water | q.s | | NA* |
| SUBTOTAL | 485 | mg | |
| Component-Tablet coating | Amount | | Weight % |
| HPMC-P | 10.0 | mg | 2.0% |
| HPMC | 5.0 | mg | 1.0% |
| Isopropyl alcohol | 0.16 | mL | NA* |
| Purified water | 0.13 | mL | NA* |
| TOTAL | 500 | mg | 100% |

*It is assumed here that the solvents (ethanol, isopropyl alcohol, and water) are present in insignificant amounts in the formulated tablet.

Coated tablets with the composition shown in Table 3 may be prepared as follows. First, zinc sulfate, α-polyglutamic acid, microcrystalline cellulose, HPMC-P (hydroxypropylmethylcellulose phthalate), maltodextrin, and carboxymethylcellulose-calcium are mixed together dry. The mixed components are transferred to a granulator and an appropriate amount of 70% aqueous ethanol is added and wet granulation was carried out. The obtained granulated mixture is dried at up to about 60° C. to yield a granulated composition with less than about 3% LOD (loss on drying). Silica (e.g., Aerosil®) and magnesium stearate is added to and mixed with the granulated composition. The obtained mixture is compressed into tablets. The tablets are first coated using an isopropyl alcohol solution of HPMC-P, and then coated in a second step using an aqueous solution of HPMC, using standard techniques, as known to those skilled in the art.

Although the invention is described herein with respect to particular embodiments, the description, examples, and illustrations should not be construed to limit the invention. Those skilled in the art will appreciate that various modifications and alterations are nonetheless with the scope of the invention.

I claim:

1. A method for treating a patient with a tumor comprising administering to said patient a therapeutically effective amount of a Zn(II) agent.

2. A method for treating a patient with a tumor comprising administering to said patient a therapeutically effective amount of a Zn(II) agent in combination with an immune-oncology agent.

3. The method according to claim 2, wherein said immune-oncology agent is an immune checkpoint inhibitor.

4. The method according to claim 3, wherein said immune checkpoint inhibitor is an anti-cytotoxic T-lymphocyte antigen-4 (CTLA-4) antibody or an antigen-binding portion thereof that binds specifically to CTLA-4 and inhibits CTLA-4 activity; or a programmed cell death-1 (PD-1) antibody or an antigen-binding portion thereof that binds specifically to a PD-1 receptor and inhibits PD-1 activity.

5. The method according to any one of claims 1 to 4, wherein said tumor includes tumor cells that have genetic instability mutations and/or genetic instability due to gene overexpression.

6. The method according to claim 5, wherein said genetic instability mutations are dysfunctional mutations in one or more genes selected from ATM; ATR; PAXIP1; BRCA1; BRCA2; WRN; RFC1; RPA1; ERCC1; ERCC4; ERCC6; MGMT; PARP1; PARP2; NEIL3; XRCC1; MLH1; PMS2; TP53; CREBBP; JAK1; NFKB1; MSH2; MSH3; MSH6; and MLH3.

7. A method for increasing the tumor infiltrating leukocyte population of CD4+ T cells and CD8+ T cells in a tumor in a patient comprising administering to said patient having said tumor a therapeutically effective amount of a Zn(II) agent.

8. A method for increasing the tumor infiltrating leukocyte population of CD4+ T cells and CD8+ T cells in a tumor in a patient comprising administering to said patient having said tumor a therapeutically effective amount of a Zn(II) agent in combination with an immune-oncology agent.

9. The method according to claim 8, wherein said immune-oncology agent is an immune checkpoint inhibitor.

10. The method according to claim 8, wherein said immune checkpoint inhibitor is an anti-cytotoxic T-lymphocyte antigen-4 (CTLA-4) antibody or an antigen-binding portion thereof that binds specifically to CTLA-4 and inhibits CTLA-4 activity; or a programmed cell death-1 (PD-1) antibody or an antigen-binding portion thereof that binds specifically to a PD-1 receptor and inhibits PD-1 activity.

11. The method according to any one of claims 1-10, wherein said Zn(II) agent comprises Zn(II)/γ-polyglutamic acid and/or Zn(II)/α-polyglutamic acid.

12. A method for treating a tumor in a patient, comprising administering a therapeutically effective amount of (i) a Zn(II)/polyglutamic acid agent in combination with (ii) an immune-oncology agent that targets a T-lymphocyte marker, a macrophage marker, or a natural killer cell marker.

13. The method of claim 12, wherein the T-lymphocyte marker is lymphocyte activation gene 3 (LAG-3).

14. The method of claim 12, wherein the T-lymphocyte marker is T-cell immunoglobulin- and mucin-domain-containing molecule 3 (TIM-3).

15. The method of claim 12, wherein the T-lymphocyte marker is T-cell immunoglobulin and ITIM domain (TIGIT).

16. The method of claim 12, wherein the T-lymphocyte marker is B7-H3 (CD276).

17. The method of claim 12, wherein the T-lymphocyte marker is V-domain containing Ig suppressor of T-cell activation (VISTA).

18. The method of claim 12, wherein the T-lymphocyte marker is inducible T-cell costimulator (ICOS).

19. The method of claim 12, wherein the T-lymphocyte marker is CD27.

20. The method of claim 12, wherein the T-lymphocyte marker is glucocorticoid-induced TNF receptor (GITR).

21. The method of claim 12, wherein the macrophage marker is CD47.

22. The method of claim 12, wherein the macrophage marker is indoleamine-2,3-dioxygenase (IDO).

23. The method of claim 12, wherein the natural killer cell marker is killer immunoglobulin-like receptor (KIR).

24. The method of claim 12, wherein the natural killer cell marker is CD94/NKG2A.

25. The method of any one of claims 12 to 24, wherein said Zn(II)/polyglutamic acid agent comprises polyglutamic acid conjugated to a tumor-targeting moiety and/or a charge-carrying moiety.

26. The method of claim 25, wherein said polyglutamic acid conjugated to a tumor-targeting moiety and/or a charge-carrying moiety is γ-polyglutamic acid.

27. The method of claim 25, wherein the molecular weight of said polyglutamic acid is in the range of about 2.5 kDa to about 60 kDa.

28. The method according to claim 5, wherein said genetic instability due to overexpression is caused by overexpression of APOBEC3B.
